(19) United States
(12) Patent Application Publication
(10) Pub. No.: US 2022/0362044 A1
(43) Pub. Date: Nov. 17, 2022

(54) DEVICES, ASSEMBLIES, AND SYSTEMS FOR DELIVERING AND DEPLOYING A GASTRIC OBSTRUCTION DEVICE AND METHODS OF OPERATION THEREOF

(71) Applicant: BAROnova, Inc., San Jose, CA (US)

(72) Inventors: David NEEDLEMAN, San Carlos, CA (US); Kobi IKI, San Carlos, CA (US)

(73) Assignee: BAROnova, Inc., San Jose, CA (US)

(21) Appl. No.: 17/321,848

(22) Filed: May 17, 2021

(51) Int. Cl. A61F 5/00

(52) U.S. Cl. CPC A61F 5/0036; A61F 5/0089; A61F 5/003

(57) ABSTRACT

Disclosed are devices, assemblies, and systems for delivering and deploying a gastric obstruction device and methods of operation thereof. A system for deploying a gastric obstruction device can comprise a housing comprising a gear mechanism; a control component coupled to the gear mechanism; a delivery tube coupled to the housing, wherein a distal end of the delivery tube is configured to be positioned within the gastric obstruction device; and a control tube coupled to the gear mechanism within the housing, wherein the control tube extends through the delivery tube lumen and is configured to engage with the gastric obstruction device, wherein the control tube is configured to rotate in response to a rotation of the control component, and wherein the rotation of the control tube is configured to rotate the gastric obstruction device.

### TECHNICAL FIELD

The present disclosure relates generally to the field of bariatrics; more specifically, to devices, assemblies, and systems for delivering and deploying a gastric obstruction device and methods of operation thereof.

### BACKGROUND

Obesity is a condition of epidemic proportions in the United States. Recent government studies have indicated that up to 40% of Americans are obese and that, among those, almost 20% are morbidly obese. Patients who are obese tend to suffer from cardiovascular disease, heart disease, stroke, diabetes, and obstructive sleep apnea. Recent studies have indicated that obesity can reduce a person's lifespan by an average of three years in adults and twenty years in children.

Many attempts have been made in the prior art to provide medications, devices, and surgical procedures for the treatment of obesity, all of which either have serious side effects or are basically ineffective. For example, various diets, supplements, and pharmaceuticals have been developed and marketed, but none have shown any significant benefits to date in the treatment of obesity with the exception of some pharmaceuticals, which have unfortunately been found to cause a number of serious, life-threatening medical conditions. To date, there are no commercially available supplements or drugs that have been proven to be effective in promoting significant weight loss and, at the same time, are free from serious collateral side effects.

Recognizing that no cure has been developed to date that is both effective and safe, the medical industry has introduced more extreme procedures, an example of which is the Roux-En-Y gastric bypass. This extensive and invasive surgery is highly effective but is also potentially lethal, with a 1-2% mortality rate, a six month recovery period, and a cost of tens of thousands of dollars, yet it is becoming increasingly popular because other available treatments do not produce the desired results. Gastric reduction, or simply removing a large segment of the stomach, is another procedure that is similar to gastric bypass and that, like gastric bypass, has also been associated with potentially lethal complications. Data from recent studies have indicated that even in the lowest risk groups, obesity surgery causes an average one-year mortality rate of nearly 5%.

In another attempt to treat obesity, devices have also been developed in the prior art that are aimed at providing a sense of fullness to a patient. Such devices may be configured as stents that support the stomach or the pyloric valve or that may be configured as permanent occluders. Unfortunately, these devices are implanted in the patient on an essentially permanent basis and typically include complex mechanical or electrical features that may stop working properly over time or that may require maintenance from time to time. Examples of such devices in the prior art can be found in U.S. Pat. Nos. 5,509,888; 6,067,991; 6,527,701; 6,689,046; 7,011,621; 7,037,344; 7,120,498; 7,122,058 and 7,167,750, and in U.S. Patent Application Publications Nos. 2004/0172142; 2005/0273060; 2007/0016262; 2007/0027548; and 2007/0083224.

A growing amount of evidence shows that benefits can be derived from reducing gastroduodenal flow. In unpublished, but recently presented data at the American Society for Bariatric Surgery Conference of June 2003, stimulation of the gastric vagus nerve with subsequent reduction in gastric motility resulted in a loss of over 20% of excess weight over a nine-month period. Furthermore, there is data suggesting that gastric vagotomy is also effective in the treatment of obesity through a similar mechanism. Unfortunately, these therapies require highly invasive, sometimes irreversible, surgical procedures, making them undesirable for a large segment of the obese population.

### SUMMARY

Disclosed are devices, assemblies, and systems for delivering and deploying a gastric obstruction device and methods of operation thereof. In one variation, a system for deploying a gastric obstruction device is disclosed. The system can be comprised of a housing comprising a gear mechanism, a control component coupled to the gear mechanism, and a delivery tube coupled to the housing. The delivery tube can have a delivery tube distal end and a delivery tube lumen therethrough. The delivery tube distal end can be configured to be positioned within the gastric obstruction device. The system can also comprise a control tube coupled to the gear mechanism within the housing. The control tube can extend through the delivery tube lumen and be configured to engage with the gastric obstruction device. The control tube can be configured to rotate in response to a rotation of the control component. The rotation of the control tube can be configured to rotate the gastric obstruction device, including a device covering of the gastric obstruction device.

The delivery tube distal end can comprise a flange. The flange can be positioned within a cavity defined by the device covering to couple the device covering to the delivery tube. The flange can be configured to seal a cover opening defined along a proximal end of the device covering. An exterior surface of the flange, an interior surface of the device covering, or a combination thereof can be covered by a polymeric coating to reduce friction between the exterior surface of the flange and the interior surface of the device covering when the device covering is rotated.

The control tube can comprise a control tube proximal segment and a control tube distal segment. The control tube distal segment can comprise a key portion compatible with a lock component positioned within the gastric obstruction device. The key portion of the control tube and the lock component of the distal hub can both comprise a similar cross-sectional shape. In one variation, the cross-sectional shape can be substantially oblong. In other variations, the cross-sectional shape can be substantially oval.

The gear mechanism can comprise a first gear component and a second gear component. The control component can be coupled to the first gear component and the control tube is coupled to the second gear component. The first gear component can be configured to rotate around a first gear axis of rotation and the second gear component can be configured to rotate around a second gear axis of rotation. The first gear axis of rotation can be perpendicular to the second gear axis of rotation.

A segment of the control tube can be configured to be bent into a curved configuration when the segment of the control tube and the delivery tube surrounding the segment of the control tube extend into a body of a patient. The control tube can be configured to rotate when in the curved configuration.

The housing can further comprise a spool and the control component can be coupled to the spool. The control tube can further comprise a control tube lumen and a plurality of tension lines extending through the control tube lumen. The rotation of the control component can be configured to reel in the tension lines extending through the control tube lumen. In addition, at least one anchor line can extend through the control tube lumen and the rotation of the control component can also reel in the anchor line extending through the control tube lumen.

In another variation, a gastric obstruction assembly is disclosed. The gastric obstruction assembly can comprise a control tube and a gastric obstruction device mated to the control tube. The control tube can comprise a control tube proximal segment and a control tube distal segment. The control tube distal segment can comprise a key portion.

The gastric obstruction device can comprise a device covering, a distal occluding member connected to the device covering by a tether extending from the device covering, and a distal hub positioned within a fillable cavity of the device covering. The device covering can also comprise a cover opening at a cover proximal end of the device covering.

The distal hub can be coupled to the device covering. The distal hub can comprise a lock component. The key portion of the control tube distal segment can be mated to the lock component of the distal hub prior to deployment of the gastric obstruction device.

The gastric obstruction device can further comprise a plurality of internal struts arranged within the fillable cavity. Each of the internal struts can be coupled to an interior surface of the device covering at one end of the internal strut and coupled to the distal hub at another end of the internal strut.

The system can further comprise a delivery tube having a delivery tube lumen. A segment of the control tube can be positioned within the delivery tube lumen and the control tube can be rotatable within the delivery tube lumen. The device covering can be configured to rotate in response to a rotation of the control tube when the key portion of the control tube distal segment is mated to the lock component of the distal hub.

The gastric obstruction device can further comprise a coil member comprising a coil proximal end and a coil distal end. The coil distal end can extend into the fillable cavity and can be detachably coupled to the distal hub. The coil member can be configured to rotate in response to a rotation of the control tube.

A method of deploying a gastric obstruction device is disclosed. The method can comprise advancing a distal segment of a delivery tube coupled to a gastric obstruction device per-orally into proximity of the stomach of a patient. A proximal segment of the delivery tube can be coupled to a housing. The method can further comprise actuating or rotating a control component coupled to the housing in a first rotational direction around a first axis of rotation when the gastric obstruction device is within the stomach of the patient. The gastric obstruction device coupled to the delivery tube can rotate in a second rotational direction around a second axis of rotation in response to the rotation of the control component. The first axis of rotation can be non-parallel to the second axis of rotation.

Actuating or rotating the control component can rotate a control tube extending through the delivery tube. The control tube can comprise a control tube proximal segment and a control tube distal segment. The control tube distal segment can be mated to the gastric obstruction device. The control tube distal segment can comprise a key portion. The gastric obstruction device can comprise a lock component. The key portion of the control tube distal segment can be mated to the lock component of the gastric obstruction device when the gastric obstruction device is rotated.

The housing can comprise a gear mechanism comprising a first gear component and a second gear component. The control component can be coupled to the first gear component and the control tube can be coupled to the second gear component. The method can further comprise actuating or rotating the control component such that the gastric obstruction device, including the device covering, is rotated at least three full rotations.

The housing can further comprise a spool coupled to the first gear component. The control tube can further comprise a control tube lumen. Actuating or rotating the control component can reel in a plurality of tension lines extending through the control tube lumen onto the spool.

The method can further comprise inflating a fillable cavity of the gastric obstruction device by delivering a fluid through a delivery tube lumen of the delivery tube into the fillable cavity.

The method can further comprise introducing a coil member into a fillable cavity of the device covering. At least a segment of the coil member can extend through a delivery tube lumen of the delivery tube prior to actuating or rotating the control component. Introduction of the coil member into the device covering can be concurrent with the rotations of the device covering and the coil member. The device covering and the coil member can both rotate in response to a rotation of the control component. The method can further comprise dislodging the device covering from the distal segment of the delivery tube when the coil member is introduced into the fillable cavity of the device covering and the coil member forms into a widened compressed configuration.

A system for deploying a gastric obstruction device is also disclosed. The system can comprise a housing comprising a worm gear, a control tube comprising a control tube proximal segment and a control tube distal segment, and a control component coupled to the worm wheel.

The worm gear can comprise a worm wheel and a worm barrel configured to rotate in response to a rotation of the worm wheel. The control tube proximal segment can be coupled to the worm barrel. The gastric obstruction device can be coupled to the control tube distal segment. The gastric obstruction device can be configured to rotate in response to a rotation of the control component.

The system can comprise a delivery tube comprising a delivery tube proximal end, a delivery tube distal end, and delivery tube lumen in between the delivery tube proximal end and the delivery tube distal end. The delivery tube can be coupled to the housing at the delivery tube proximal end and the delivery tube distal end can be configured to couple to the gastric obstruction device.

At least a segment of the control tube can be configured to extend through the delivery tube lumen. Each of the delivery tube and the control tube can be bendable into a curved configuration. The control tube can be configured to rotate when in the curved configuration within the delivery tube.

The control tube can be fabricated from a biocompatible polymeric material. The worm wheel can comprise a plurality of wheel blades that extend radially outward from a circumferential surface of a wheel disk. The worm barrel can comprise a plurality of barrel grooves that project radially inward from a lateral surface of the worm barrel to define a plurality of groove surfaces. The rotation of the worm wheel can cause at least one of the wheel blades to impart a translational motion to at least one of the groove surfaces to rotate the worm barrel.

Each of the wheel blades can have a blade ridge and the blade ridge can be aligned at an oblique angle relative to a midline bisecting the circumferential surface of the wheel disk. A length dimension of each of the wheel blades can be less than a circumference of the wheel disk.

The worm barrel can comprise a barrel proximal portion and a barrel distal portion. The worm barrel can further comprise a plurality of barrel grooves that project radially inward from a lateral surface of the worm barrel. Each of the barrel grooves can be oriented substantially in a longitudinal direction such that each of the barrel grooves extends from the barrel proximal portion to the barrel distal portion.

The worm barrel can comprise a radially convergent midsection. The worm barrel can be configured to be rotated 360 degrees in response to a rotation of the worm wheel of 1080 degrees. The worm barrel can comprise a barrel lumen and a segment of the control tube can extend into the worm barrel.

The housing can further comprise a spool and the worm wheel can be coupled to the spool. The control tube can further comprise a control tube lumen and a plurality of tension lines extending through the control tube lumen. The rotation of the control component can be configured to reel in the tension lines extending through the control tube lumen.

A method of deploying a gastric obstruction device is disclosed. The method can comprise advancing the gastric obstruction device per-orally into proximity of the stomach of a patient and actuating or rotating a control component coupled to a worm wheel of the worm gear.

The gastric obstruction device can be coupled to a distal end of a control tube. A proximal end of the control tube can be coupled to a worm barrel of a worm gear. The worm wheel can be rotated in response to the rotation of the control component. The worm barrel can be rotated in response to the rotation of the worm wheel. The gastric obstruction device can be rotated within the stomach of the patient in response to the rotation of the worm wheel.

The worm wheel can rotate around a first gear axis of rotation. The worm barrel can rotate around a second gear axis of rotation. The first gear axis of rotation can be substantially perpendicular to the second gear axis of rotation.

The method can further comprise actuating or rotating the control component at least nine full rotations. In other variations, the method can comprise actuating or rotating the control component between six and nine full rotations.

The method can further comprise bending the control tube into a curved configuration in order to advance the gastric obstruction device per-orally into the stomach of the patient. The control tube can be rotated in response to the rotation of the worm barrel. The control tube can be in the curved configuration when rotated.

The method can further comprise coupling the control tube to the gastric obstruction device by mating a key portion of a control tube distal segment with a lock component within the gastric obstruction device. The gastric obstruction device can further comprise a device covering coupled to the lock component. The device covering can rotate in response to the rotation of the control component. The gastric obstruction device can further comprise a coil member detachably coupled to the device covering. The coil member can rotate in response to the rotation of the control component. The method can further comprise retracting the control tube from the gastric obstruction device prior to removing the control tube from the esophagus of the patient.

A method of preparing and/or operating a gastric obstruction device is disclosed. The method can comprise providing a distal segment of a delivery tube coupled to a gastric obstruction device. A proximal segment of the delivery tube can be coupled to a housing. The method can further comprise actuating a control component coupled to the housing in a first rotational direction around a first axis of rotation. The gastric obstruction device coupled to the delivery tube can rotate in a second rotational direction around a second axis of rotation in response to the rotation of the control component. The first axis of rotation can be non-parallel to the second axis of rotation.

Another method of preparing and/or operating a gastric obstruction device is also disclosed. The method can comprise providing the gastric obstruction device. The gastric obstruction device can be coupled to a distal end of a control tube. A proximal end of the control tube can be coupled to a worm barrel of a worm gear. The method can further comprise actuating a control component coupled to a worm wheel of the worm gear. The worm wheel can be rotated in response to the rotation of the control component. The worm barrel can be rotated in response to the rotation of the worm wheel. The gastric obstruction device can be rotated in response to the rotation of the worm wheel.

### BRIEF DESCRIPTION OF THE DRAWINGS

FIG. 1 illustrates a perspective view of one variation of a system for deploying a gastric obstruction device.

FIGS. 2A and 2B illustrate a variation of the gastric obstruction device deployed within a stomach of a patient.

FIG. 3 illustrates an exploded view of a variation of the gastric obstruction device and a part of the system for deploying the gastric obstruction device.

FIGS. 4A and 4B illustrate perspective and side cross-sectional views, respectively, of a variation of the gastric obstruction device in a deployed state.

FIG. 4C illustrates a perspective cross-sectional view of a part of the coil member locked into a contracted widened configuration.

FIG. 5A illustrates the coil member in a relaxed state.

FIG. 5B illustrates the coil member in an elongated configuration.

FIG. 5C illustrates the coil member in a partially unwound configuration.

FIG. 6 is a black-and-white image of a variation of the gastric obstruction device coupled to a delivery tube and an elongated and partially unwound coil member positioned within the delivery tube.

FIG. 7 illustrates a perspective view of one variation of part of a system for deploying the gastric obstruction device.

FIG. 8A illustrates a perspective cutaway view of a distal hub positioned within a device covering.

FIG. 8B illustrates a top plan view of the distal hub positioned within the device covering shown in FIG. 8A.

FIG. 9A illustrates a perspective view of a distal segment of a variation of a control tube.

FIG. 9B illustrates a top plan view of the distal segment of the variation of the control tube shown in FIG. 9A.

FIG. 10 illustrates a variation of a gear mechanism of the system for deploying the gastric obstruction device.

FIG. 11A illustrates a front view of a first gear component operatively engaged with a second gear component of part of the gear mechanism shown in FIG. 10.

FIG. 11B illustrates a side view of the first gear component operatively engaged with the second gear component of part of the gear mechanism shown in FIG. 10.

FIG. 12A illustrates a side cross-sectional view of a variation of a flange uncoupled to the device covering of the gastric obstruction device.

FIG. 12B illustrates a side cross-sectional view of the flange coupled to a device covering of the gastric obstruction device.

FIG. 13 is a black-and-white image of a variation of part of the gastric obstruction device comprising a device covering, a tether, and a distal occluding member.

FIG. 14A is a black-and-white image of tension lines extended through helically wound coils of a coil member and into a distal hub of the gastric obstruction device.

FIG. 14B is a black-and-white image of tension lines passing through a distal hub into a lumen of a control tube at a distal end of the control tube.

FIG. 14C is a black-and-white image of tension lines exiting a lumen of the control tube at a proximal end of the control tube.

FIG. 15 illustrates a variation of a spool configured to rotate in response to a rotation of a control component.

FIG. 16 is a black-and-white image of a plurality of tension lines and an anchor line extending into a distal hub of the gastric obstruction device.

FIG. 17 is a black-and-white image of a pair of tension lines coupled to lock lines.

FIG. 18A illustrates a variation of a plunger translated distally through a lumen of the delivery tube as part of the deployment of the gastric obstruction device.

FIG. 18B illustrates the coil member locked in a contracted widened configuration within a fillable cavity of a device covering of the gastric obstruction device.

FIG. 18C illustrates a separation of the delivery tube from the deployed gastric obstruction device.

FIG. 19 illustrates a perspective view of another variation of a system for deploying a gastric obstruction device.

### DETAILED DESCRIPTION

Detailed descriptions of embodiments of the invention are provided herein. It is to be understood, however, that the present invention may be embodied in various forms. Therefore, the specific details disclosed herein are not to be interpreted as limiting, but rather as a representative basis for teaching one skilled in the art how to employ the present invention in virtually any detailed system, structure, or manner.

The devices, assemblies, systems, and methods disclosed herein are suited not only for the treatment of obesity but also for treating other ailments, such as improper glucose tolerance in a diabetic or pre-diabetic subject and the progression of diabetes itself by inhibiting fasting insulin secretion or glucose-stimulated insulin secretion. The device of the present disclosure is also suited for treating other ailments deriving from obesity, including hyperphagia, dyslipidemia, Prader Willi syndrome, Froelich's syndrome, Cohen syndrome, Summit syndrome, Alstrom syndrome, Borjesen syndrome, Bardet-Biedl syndrome, or hyperlipoproteinemia, types I, II, III, and IV, etc. The devices, assemblies, and systems disclosed herein are well tolerated by the stomach and in general, by the gastrointestinal tract. The devices, assemblies, and systems disclosed herein can be introduced or implanted and removed with medical procedures that are safe and relatively simple to perform.

The gastric obstruction device disclosed herein can include sensors or transmitters to provide feedback and other data to an intra-corporeal or extra-corporeal processor, or may carry one or more compounds stored in a reservoir within the device or coated on the device. In some variations, insulin is released into the gastrointestinal tract by disposing an insulin reservoir in the distal occluding member of the device. Such a release of insulin may be controlled by the size of the orifice between the reservoir and the outer environment, or by a time-controlled actuator, or by an actuator controlled by one or more sensors, for example in response to detection of sugar in the gastrointestinal tract.

Additionally, the devices, assemblies, systems, and methods disclosed herein can also be compatible and/or substituted with any of the devices, assemblies, systems, and methods disclosed in U.S. patent application Ser. No. 12/205,403 filed on Sep. 5, 2008 (U.S. Patent Publication No. 2009/0198210); U.S. patent application Ser. No. 12/352,497 filed on Jan. 12, 2009 (U.S. Patent Publication No. 2009/0182357); U.S. patent application Ser. No. 12/352,508 filed on Jan. 12, 2009 (U.S. Patent Publication No. 2009/0182358); and U.S. patent application Ser. No. 15/878,319 filed on Jan. 23, 2018, each of which is incorporated herein by reference in its entirety for any purpose.

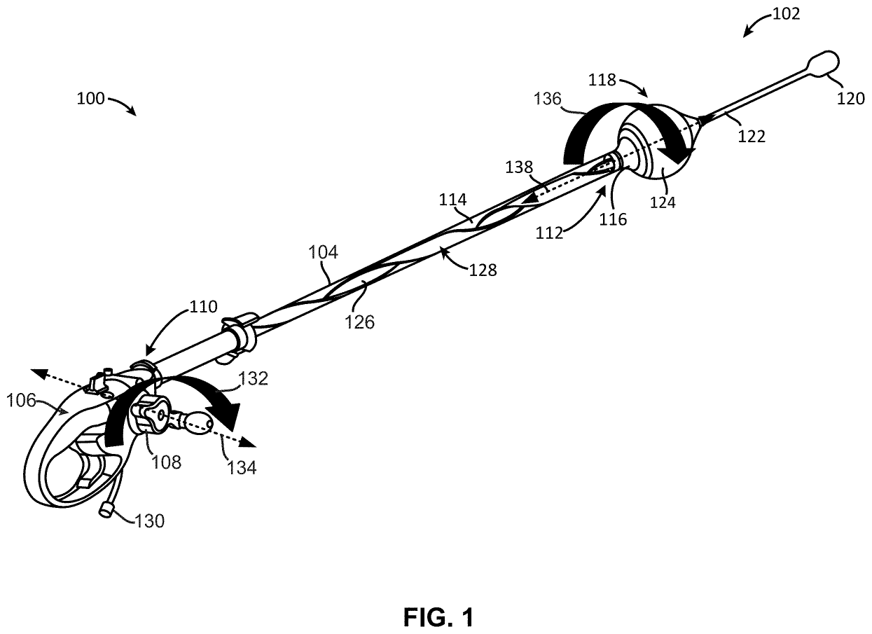

FIG. 1 illustrates a perspective view of one variation of a system 100 for deploying a gastric obstruction device 102. The system 100 can comprise a delivery tube 104 coupled to a housing 106. Although FIG. 1 depicts the housing 106 as part of a handle, the housing 106 can also refer to a part of a stationary device, a benchtop or desk top device, a portable device comprising wheels, a winch housing, a gear box, or a combination thereof. The system 100 can also comprise a control component 108 coupled to the housing 106. FIG. 1 illustrates that the control component 108 can have a hand crank coupled to or integrated with the control component 108. In some variations, the control component 108 can be a control knob, an advance knob, a wing knob, a lobed knob, a dimple knob, a cross-shaped knob, a triangular knob, a ball knob, or a combination thereof. The control component 108 can be coupled to a lateral or side surface of the housing 106.

The delivery tube 104 can comprise a delivery tube proximal end 110, a delivery tube distal end 112, and a delivery tube lumen 114. The delivery tube 104 can be fabricated from or be composed of a biocompatible or medical-grade polymeric material. For example, the delivery tube 104 can be fabricated from or be composed of polytetrafluoroethylene (PTFE), silicone, medical-grade polyvinyl chloride, or a combination thereof. In other variations, the delivery tube 104 can be fabricated from or be composed of a thin-walled metallic material. In some variations, the walls of the delivery tube 104 can be translucent or see-through such that contents within the delivery tube lumen 114 are visible from the outside of the delivery tube 104.

The delivery tube 104 can further comprise a flange 116 positioned at the delivery tube distal end 112. The flange 116 can be used, along with other components, to secure part of the gastric obstruction device 102 to the delivery tube 104. The flange 116 will be discussed in more detail in the following sections.

The gastric obstruction device 102 can comprise a proximal occluding member 118 coupled to a distal occluding member 120 by a tether 122. The distal occluding member 120 can be shaped and sized for passage through the pylorus of a patient when the gastric obstruction device 102 is deployed within the stomach of the patient. Deployment of the gastric obstruction device 102 will be discussed in more detail in the following sections.

The distal occluding member 120 can be substantially shaped as an ellipsoid, an ovoid, a combination thereof, or any number of other atraumatic shapes. The distal occluding member 120 can have a maximum cross-sectional diameter of between approximately 14 mm and 18 mm. In some variations, the distal occluding member 120 can have a maximum cross-sectional diameter of between approximately 15 mm and 16 mm.

The tether 122 can be fabricated from or be composed of silicone, silicone rubber, urethanes, a thermoplastic elastomer, copolymers thereof, or a combination thereof. The tether 122 can have elastic properties such that the length of the tether 122 can vary as the tether 122 is stretched or allowed to contract.

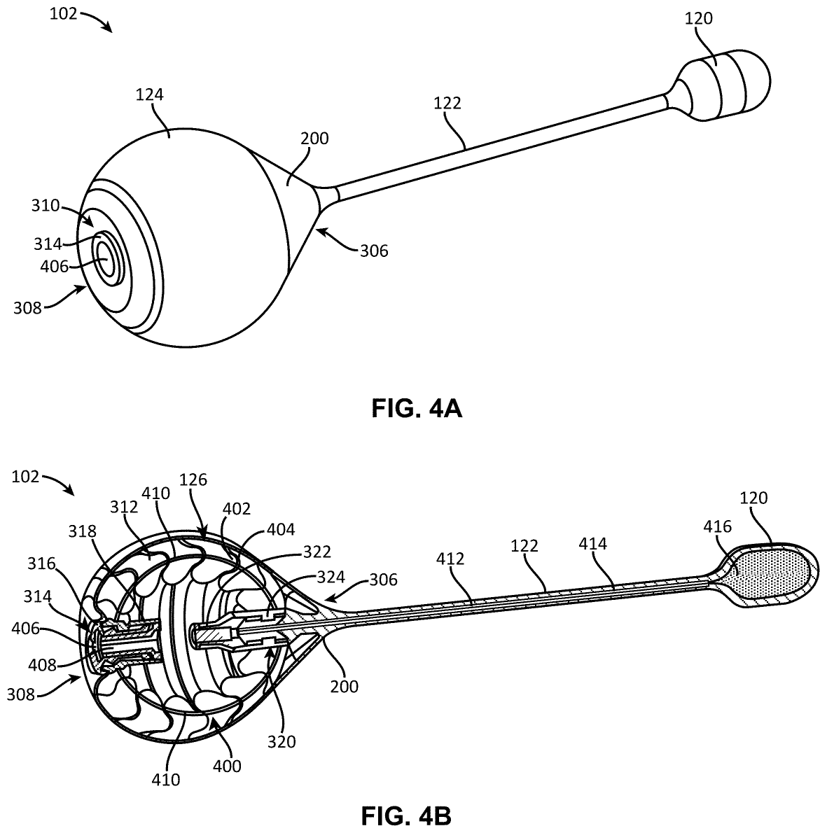

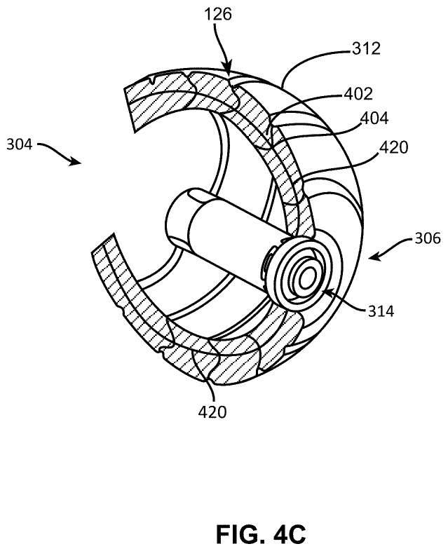

The proximal occluding member 118 can comprise a device covering 124 surrounding and encapsulating a fillable cavity 400 (see FIG. 4B). The proximal occluding member 118 can further comprise a coil member 126. A segment of the coil member 126 can be positioned within the delivery tube 104 when the device covering 124 is secured to the delivery tube distal end 112 by the flange 116.

As depicted in FIG. 1, the coil member 126 can be extended into an elongated narrow configuration 128 such that the entire coil member 126 is stretched longitudinally and the cross-sectional size of the coil member 126 is reduced. As shown in FIG. 1, the coil member 126 can fit within a confined space such as the delivery tube lumen 114 when in the elongated narrow configuration 128.

In some variations, the tether 122 can be integrated with the device covering 124 such that the tether 122 is an extension of the device covering 124. The device covering 124 and the coil member 126 will be discussed in more detail in the following sections.

The system 100 can also comprise a fluid delivery port 130 or tube extending from the housing 106. A gas or fluid can be introduced into the fluid delivery port 130 to inflate a part of the gastric obstruction device 102 (e.g., the device covering 124) and to insufflate the stomach of a patient receiving the gastric obstruction device 102.

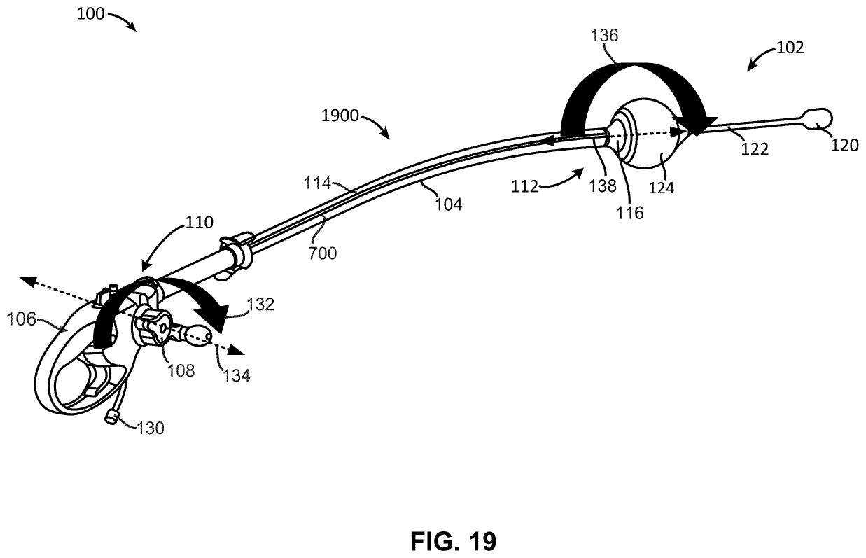

FIG. 1 also illustrates that the control component 108 can be rotated in a first rotational direction 132. The control component 108 can be rotated in the first rotational direction 132 around a first axis of rotation 134 when at least part of the gastric obstruction device 102 is within the stomach of the patient. The gastric obstruction device 102 coupled to the delivery tube distal end 112 can rotate in a second rotational direction 136 around a second axis of rotation 138 in response to the rotation of the control component 108 in the first rotational direction 132. The first axis of rotation 134 can be non-parallel to the second axis of rotation 138. The first axis of rotation 134 can be non-parallel to the second axis of rotation 138 when the first axis of rotation 134 is either perpendicular or oblique (e.g., obtuse or acute) to the second axis of rotation 138. For example, the first axis of rotation 134 can be substantially perpendicular to the second axis of rotation 138 when the delivery tube 104 is kept substantially straight. As a more specific example, the first axis of rotation 134 can be a lateral axis extending from one lateral side of the housing 106 to another lateral side of the housing 106. In this example, when the delivery tube 104 is kept straight, the second axis of rotation 138 can be a longitudinal axis running along the length of the delivery tube 104. As will be discussed in the following sections, when the delivery tube 104 is in a curved configuration 1900 (see FIG. 19), the first axis of rotation 134 can be oblique (e.g., obtuse or acute) to the second axis of rotation 138 or the first axis of rotation 134 can meet the second axis of rotation 138 at an oblique angle (e.g., obtuse or acute angle). The delivery tube 104 can be bent into the curved configuration 1900 when at least part of the delivery tube 104 is extended into the esophagus of a patient through the oral cavity (or mouth) and pharynx (or throat) of the patient.

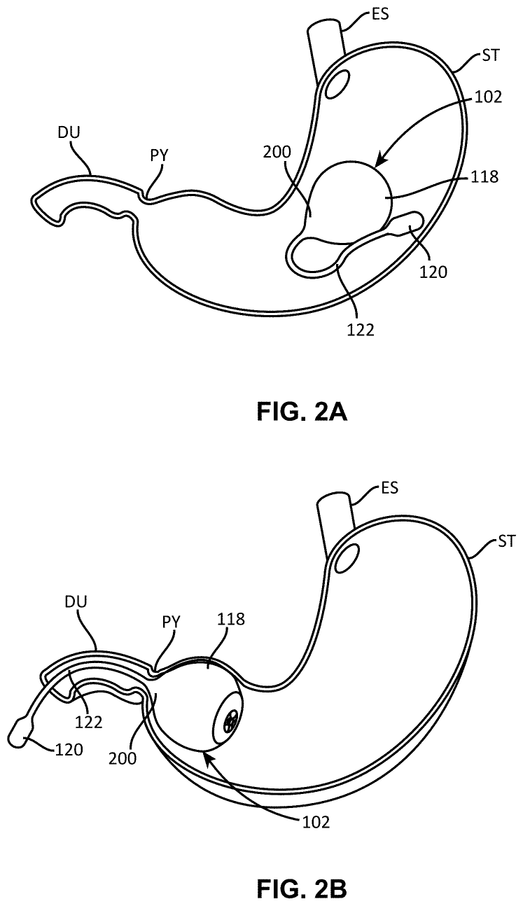

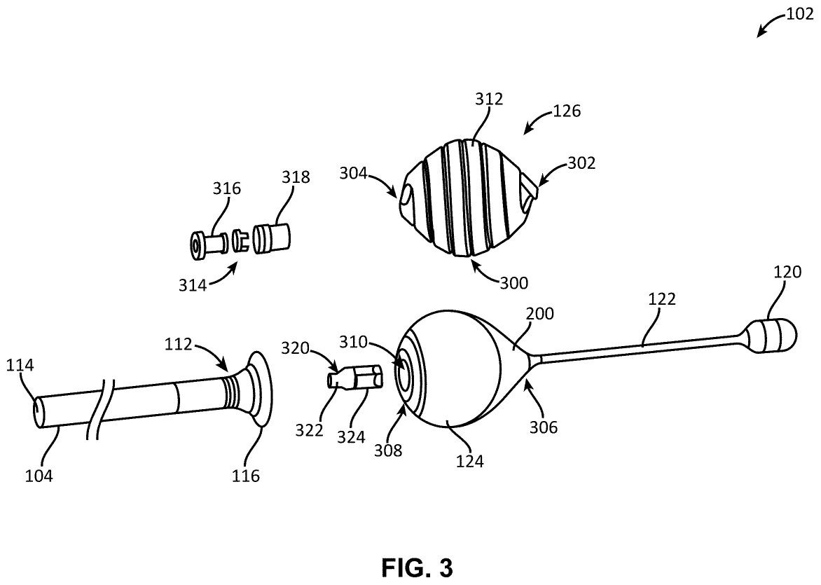

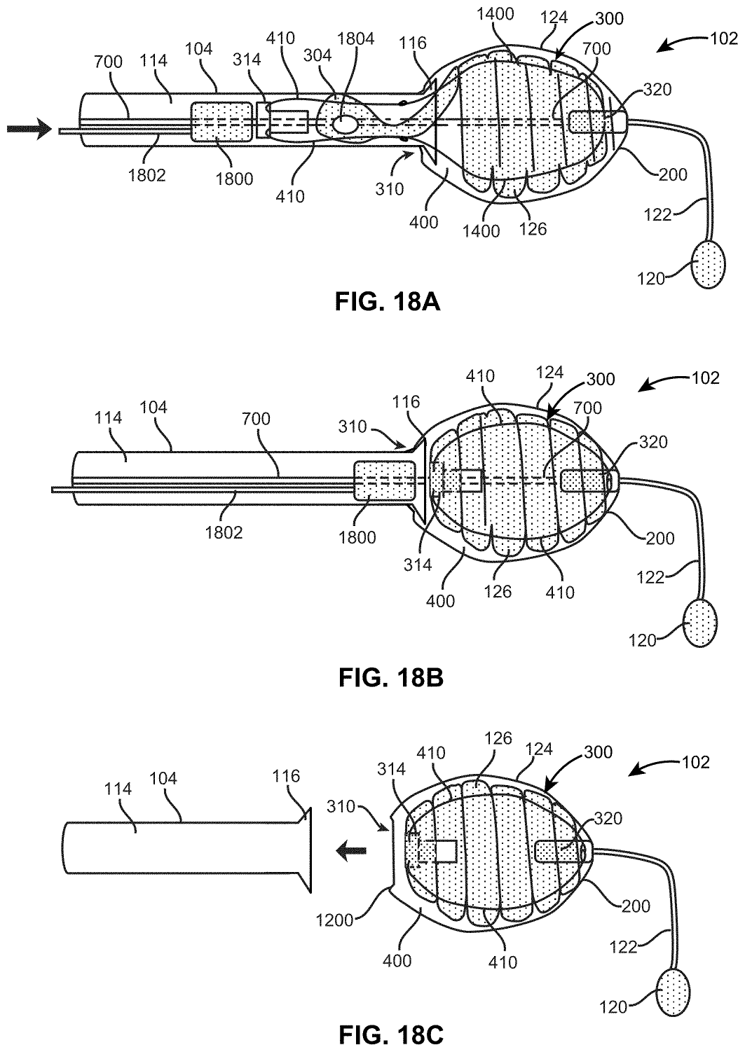

FIGS. 2A and 2B illustrate a variation of the gastric obstruction device 102 deployed within the stomach (shown as ST in FIGS. 2A and 2B) of a patient. The esophagus (shown as ES), pylorus (shown as PY) and duodenum (shown as DU) of the patient are also depicted in FIGS. 2A and 2B for reference. The gastric obstruction device 102 can be fully deployed within the stomach of the patient when the coil member 126 (see FIG. 1) of the proximal occluding member 118 is formed or configured from the elongated narrow configuration 128 (see FIG. 1) into a contracted widened configuration 300 (see FIG. 3, FIG. 4B, and FIG. 18C). As will be discussed in the following sections, the coil member 126 can be locked into the contracted widened configuration 300 within the device covering 124 of the proximal occluding member 118.

Once the patient has ingested food or liquids, the stomach can begin to contract and relax, repeatedly, such that the distal occluding member 120 is propelled or otherwise moved by peristaltic waves through the stomach towards the pylorus. As is shown in FIG. 2B, the distal occluding member 120 can be shaped and sized for passage through the pylorus and the distal occluding member 120, the tether 122, or a combination thereof can be positioned within the duodenum of the patient. Due to the size and substantially spherical shape of the proximal occluding member 118, the proximal occluding member 118 is unable to pass through the pylorus and remains within the stomach.

As illustrated in FIGS. 2A and 2B, the proximal occluding member 118 can comprise a tapered pyloric contact region 200 in proximity to the tether 122 of the gastric obstruction device 102. The tapered pyloric contact region 200 can be tapered to narrow from a larger cross-sectional diameter to a smaller cross-sectional diameter toward a distal end of the proximal occluding member 118. In some variations, the tapered pyloric contact region 200 can refer to a segment or section of the device covering 124 in proximity to the tether 122. In other variations, the tapered pyloric contact region 200 can refer to a portion of the proximal occluding member 118 coupled to the device covering 124.

The tapered pyloric contact region 200 can be substantially conical or frustoconical in shape. In some variations, the tapered pyloric contact region 200 can comprise a taper angle of between approximately 30° and 50° with respect to a longitudinal axis of the gastric obstruction device 102. The tapered pyloric contact region 200 can be compliant or compressible.

The tapered pyloric contact region 200 of the gastric obstruction device 102 can intermittently cover or obstruct the pylorus as shown in FIG. 2B. In addition, the tapered pyloric contact region 200 can also intermittently expose and allow food or liquids to pass through the pylorus as the stomach and other digestive organs of the patient relax. This intermittent obstruction of the pylorus can cause food and/or liquids to pass from the stomach into the duodenum at a slower rate, thus inducing the patient to feel full sooner and reduce the patient's craving for more food. Once the stomach has been completely emptied, the gastric obstruction device 102 can reposition itself within the stomach of the patient.

Although not shown in FIGS. 2A and 2B, it is contemplated by this disclosure that the gastric obstruction device 102 can be removed from the patient. The gastric obstruction device 102 can be removed by being collapsed and removed back through the esophagus of the patient. An access sheath, overtube, or a combination thereof can be positioned within the esophagus of a patient and a grasping tool and endoscope can be passed through the access sheath into proximity with the proximal occluding member 118 of the gastric obstruction device 102. The grasping tool can be brought into contact with a release mechanism 316 (see FIG. 3) of the proximal occluding member 118 and actuation of the release mechanism 316 can cause the coil member 126 to transform or re-configure from the contracted widened configuration 300 (see FIG. 3, FIG. 4B, and FIG. 19C) into the elongated narrow configuration 128 (see FIG. 1). With the coil member 126 in the elongated narrow configuration 128, the device covering 124 of the proximal occluding member 118 can be collapsed or compressed so as to fit through the access sheath. The coil member 126, the device covering 124, the tether 122, and the distal occluding member 120 can then be pulled through the access sheath and removed from the stomach through the esophagus, pharynx, and oral cavity of the patient.

FIG. 3 illustrates an exploded view of a variation of the gastric obstruction device 102 and a part of the system 100 for deploying the gastric obstruction device 102. As depicted in FIG. 3, the coil member 126 can be compressed or otherwise formed into a contracted widened configuration 300. The coil member 126 can be compressed or otherwise formed into the contracted widened configuration 300 within a fillable cavity 400 (see FIG. 4B) of the device covering 124.

The coil member 126 can comprise a coil distal end 302 and a coil proximal end 304. The coil distal end 302 can be initially positioned within the delivery tube 104 when the device covering 124 is detachably coupled to the delivery tube distal end 112 by the flange 116. In some variations, the coil distal end 302 can be positioned within the fillable cavity 400 (see FIG. 4B) of the device covering 124 when the device covering 124 is detachably coupled to the delivery tube distal end 112. In other variations, the coil distal end 302 can be initially positioned within the delivery tube lumen 114 and introduced into the fillable cavity 400 during the deployment procedure.

FIG. 3 also illustrates that the device covering 124 can comprise a cover distal end 306 and a cover proximal end 308. The device covering 124 can also have a cover opening 310 at the cover proximal end 308. The cover opening 310 can be a substantially circular-shaped void or aperture at the cover proximal end 308. The coil member 126 can be introduced into the fillable cavity 400 (see FIG. 4B) of the device covering 124 through the cover opening 310. The device covering 124 can be fabricated from or be composed of silicone, silicone rubber, urethanes, a thermoplastic elastomer, copolymers thereof, or a combination thereof.

As depicted in FIG. 3, the coil member 126 can comprise a number of helically wound coils 312 or loops. The helically wound coils 312 or loops can physically be pressed against one another when the coil member 126 is in the contracted widened configuration 300. As will be discussed in the following sections, a portion of each of the helically wound coils 312 or loops can be nested within at least one neighboring helically wound coil 312 or loop when the coil member 126 is in the contracted widened configuration 300.

In one variation, the coil member 126 can comprise six helically wound coils 312 or loops. In other variations, the coil member 126 can comprise between approximately five and eight helically wound coils 312 or loops. The cross-sectional diameter or coil diameter of the median or middle coil or loop can be the greatest among all of the coils or loops when the coil member 126 is in the contracted widened configuration 300.

FIG. 3 also illustrates that the gastric obstruction device 102 can comprise a proximal assembly 314 coupled to the coil proximal end 304 of the coil member 126. In one variation, the proximal assembly 314 can be a substantially columnar-shaped component comprising a release mechanism 316 and a proximal plug 318. The release mechanism 316 can extend partially into the proximal plug 318 and be coupled to the proximal plug 318 by an interference fit. In this and other variations, the release mechanism 316 can also be coupled to the proximal plug 318 by fasteners, adhesives, a connecting member or string, or a combination thereof. In one variation, the proximal assembly 314 can be coupled to the coil proximal end 304 of the coil member 126. In other variations, the proximal assembly 314 can be positioned within a loop at the coil proximal end 304. The proximal assembly 314 can function as a cap or bung to partially occlude or constrict an opening at the coil proximal end 304 when the coil member 126 is in the contracted widened configuration 300. For example, the proximal assembly 314 can partially occlude or constrict the opening at the coil proximal end 304 in order to impede or hinder fluids or particles from entering into a coil lumen created by the wound coil member 126. As will be discussed in the following sections, the proximal assembly 314 can serve as one part of a locking mechanism to lock the coil member 126 in the contracted widened configuration 300.

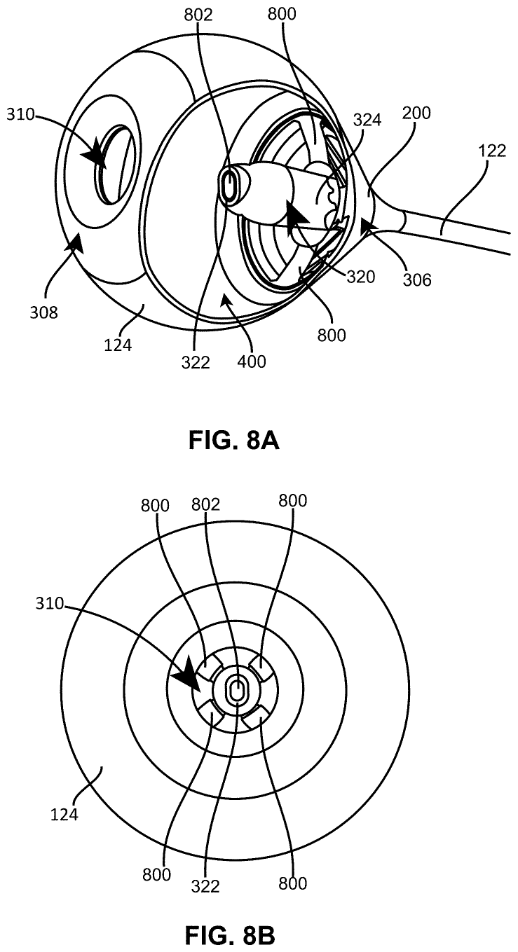

FIG. 3 illustrates that the gastric obstruction device 102 can also comprise a distal hub 320. The distal hub 320 can further comprise a lock component 322 and an attachment collar 324 coupled to the lock component 322. In one variation, the lock component 322 can fit within the attachment collar 324 by an interference fit. In this and other variations, the lock component 322 can also be coupled to the attachment collar 324 by adhesives, fasteners, or a combination thereof. The distal hub 320 can be positioned within the fillable cavity 400 (see FIG. 4B) of the device covering 124. As will be discussed in more detail in the following sections, the distal hub 320 can be coupled to the device covering 124 by internal struts 800 (see FIGS. 8A and 8B) within the device covering 124. The distal hub 320 can also be used with the proximal assembly 314 to lock the gastric obstruction device 102 into the contracted widened configuration 300.

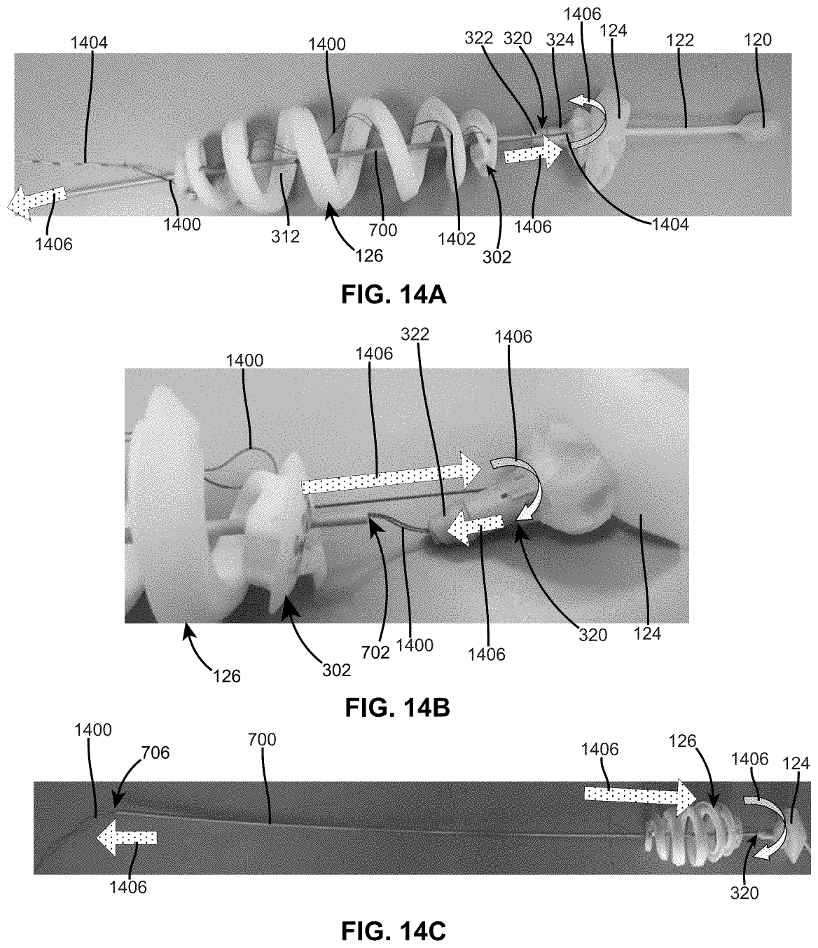

In some variations, the coil distal end 302 of the coil member 126 can be initially positioned within the delivery tube lumen 114 and pulled or otherwise advanced into the fillable cavity 400 (see FIG. 4B) of the device covering 124 by tension lines 1400 (see FIGS. 14A, 14B, and 14C). In these and other variations, the coil distal end 302 can be positioned around a portion of the distal hub 320 and abut or press against an internal surface of the cover distal end 306. Moreover, the coil distal end 302 of the coil member 126 can be fitted or otherwise coupled to the distal hub 320 such that any rotation of the distal hub 320 results in or translates into a rotation of the coil member 126. Rotation of the distal hub 320 will be discussed in more detail in the following sections.

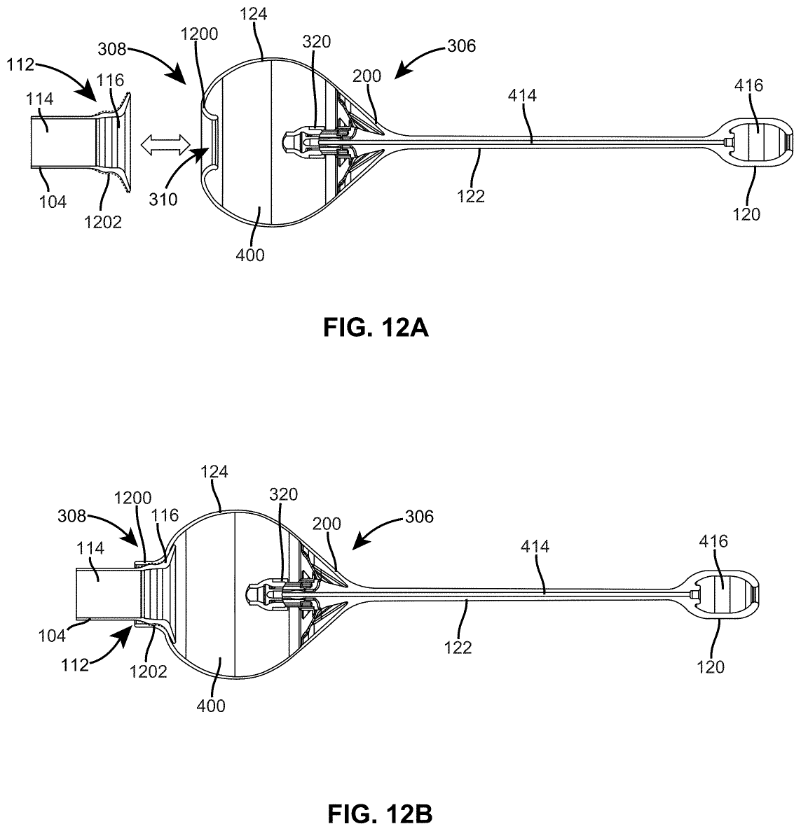

FIG. 3 also illustrates that the flange 116 of the delivery tube 104 can extend through the cover opening 310 of the device covering 124 and detachably secure the device covering 124 to the delivery tube distal end 112. For example, the flange 116 can be momentarily compressed or constricted when it enters through the cover opening 310 and then expand when at least part of the flange 116 is positioned within the fillable cavity 400 (see FIG. 12B). As will be discussed in the following sections, an inwardly curving portion 1200 (see FIG. 12A) of the device covering 124 surrounding the cover opening 310 can also be biased outward by the flange 116 and the radially inward force exerted by this previously inwardly curving portion on the flange 116 can facilitate the coupling or attachment of the device covering 124 to the delivery tube 104. In addition, a part of the flange 116 within the fillable cavity 400 can also exert an outwardly expanding force on the portion of the device covering 124 surrounding and in proximity to the cover opening 310.

FIGS. 4A and 4B illustrate perspective and side cross-sectional views, respectively, of a variation of the gastric obstruction device 102 in a deployed state. FIGS. 4A and 4B illustrate that a fillable cavity 400 within the device covering 124 can be filled by the coil member 126 locked or otherwise formed into the contracted widened configuration 300. As depicted in FIG. 4B, the coil member 126 can have a number of coil protuberances 402 and coil furrows 404 defined along a length of the coil member 126. The coil protuberances 402 can fit within the coil furrows 404 such that each of the helically wound coils 312 can partially physically nest within one neighboring helically wound coil 312 when the coil member 126 is formed into the contracted widened configuration 300. For example, the coil furrows 404 can be defined along an underside of the coil member 126 and the coil protuberances 402 can be defined along a top side of the coil member 126 when viewing the coil member 126 from the coil distal end 302 to the coil proximal end 304. As depicted in FIG. 4A, the device covering 124 can assume a bulbous shape, a teardrop shape, or a substantially spherical shape having a tapered end when the coil member 126 in the contracted widened configuration 300 is fully encapsulated or surrounded by the device covering 124.

FIGS. 4A and 4B illustrate that the proximal assembly 314 can extend into the cover opening 310 and at least part of the proximal assembly 314 can be surrounded or encircled by a number of the helically wound coils 312. FIGS. 4A and 4B illustrate that the proximal assembly 314 can comprise a proximal assembly opening 406 and a proximal assembly lumen 408.

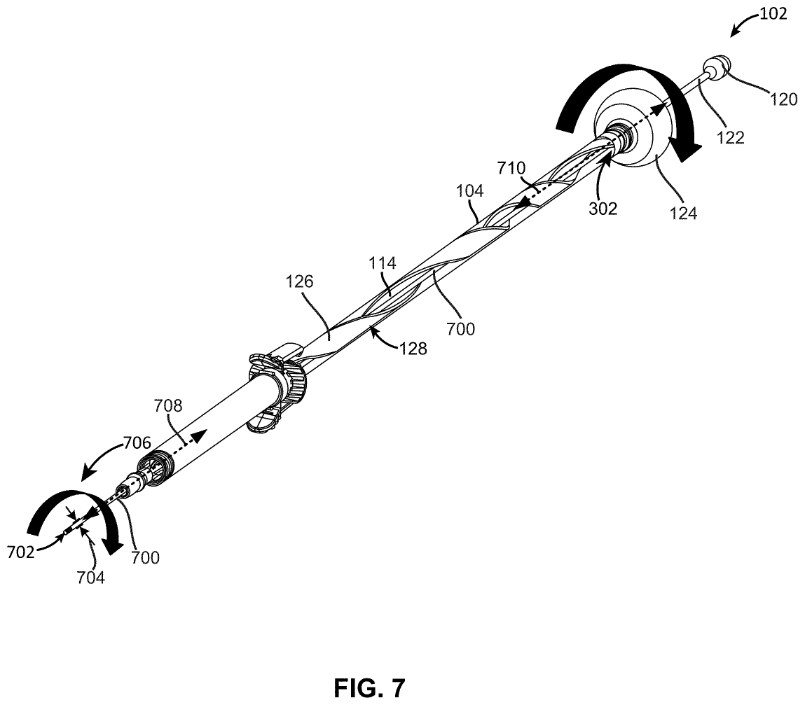

The proximal assembly opening 406 and the proximal assembly lumen 408 can allow a control tube 700 (see FIG. 7) to extend into a lumen created by the helically wound coils 312 and contact the lock component 322 of the distal hub 320. As will be discussed in more detail in the following sections, a segment of the control tube 700 (see FIG. 7) can extend through the proximal assembly opening 406 and the proximal assembly lumen 408 and mate with the lock component 322 of the distal hub 320.

FIG. 4B also illustrates that a plurality of lock lines 410 can extend or pass through the helically wound coils 312 of the coil member 126. For example, as shown in FIG. 4B, multiple pairs of lock lines 410 can extend or pass through the helically wound coils 312 in a curved trajectory, a longitudinal trajectory, or a combination thereof. The lock lines 410 can extend or pass through bores, openings, or channels defined transversely through each of the helically wound coils 312. The lock lines 410, the proximal assembly 314, and the distal hub 320 can be configured to lock the coil member 126 in the contracted widened configuration 300. Tension can be applied to the lock lines 410 such that the lock lines 410 are taut when the coil member 126 is locked in the contracted widened configuration 300. As will be discussed in the following sections, the lock lines 410 can be pulled into position by one or more tension lines 1400 (see FIGS. 14A, 14B, and 14C). Although two pairs of lock lines 410 are shown in FIG. 4B, it is contemplated by this disclosure that between four and eight pairs of lock lines 410 can be arranged uniformly around a circumference of the coil member 126 when the coil member 126 is in the contracted widened configuration 300 (e.g., four pairs of lock lines 410 can be positioned substantially at 90° relative to one another about a circumference of each of the helically wound coils 312).

FIG. 4B also illustrates that the tether 122 can comprise a tether lumen 412. In one variation, a reinforcing member 414, such as a wire, suture, string, or a combination thereof, can extend through the tether lumen 412. The reinforcing member 414 can be coupled at one end to a component within the proximal occluding member 118 (e.g., the distal hub 320) and be coupled at another end to the distal occluding member 120. The reinforcing member 414 can serve to prevent over-extension of the tether 122 during deployment and use of the gastric obstruction device 102. In addition, the reinforcing member 414 can prevent the detachment of the distal occluding member 120 from the proximal occluding member 118 in the unlikely event that the tether 122 fails.

FIG. 4B also illustrates that the distal occluding member 120 can comprise a distal weight 416. The distal weight 416 can be a separate component encapsulated by the distal occluding member 120 or be integrated with the distal occluding member 120. The distal weight 416 can be fabricated from or be composed of a metallic material, a polymeric material, or a combination thereof. The distal weight 416 can function to weigh down the distal occluding member 120 and facilitate disposition and retention of the distal occluding member 120 within the duodenum.

FIG. 4C illustrates a perspective cross-sectional view of part of the coil member 126 locked into the contracted widened configuration 300. For example, the coil member 126 can be locked into the contracted widened configuration 300 within a fillable cavity 400 (see FIG. 8A) of the device covering 124. FIG. 4C also illustrates that the coil member 126 can have coil protuberances 402 and coil furrows 404 defined along a length of the coil member 126. The coil protuberances 402 can fit within the coil furrows 404 such that each of the helically wound coils 312 can partially nest within a neighboring helically wound coil 312 when the coil member 126 is formed into the contracted widened configuration 300. The coil furrows 404 can be defined along an underside of the coil member 126 and the coil protuberances 402 can be defined along a top side of the coil member 126 when viewing the contracted coil member 126 from the coil distal end 302 to the coil proximal end 304.

In addition, a plurality of lock lines 410 can extend or pass through the helically wound coils 312 in a curved trajectory, a longitudinal trajectory, or a combination thereof. The lock lines 410 can extend or pass through bores, openings, or channels defined transversely through each of the helically wound coils 312. The lock lines 410 can facilitate the locking of the coil member 126 in the contracted widened configuration 300. As will be discussed in the following sections, the lock lines 410 can be pulled into position by one or more tension lines 1400 (see FIGS. 14A, 14B, and 14C). The lock lines 410 can be arranged uniformly around a circumference of the coil member 126 when the coil member 126 is in the contracted widened configuration 300.

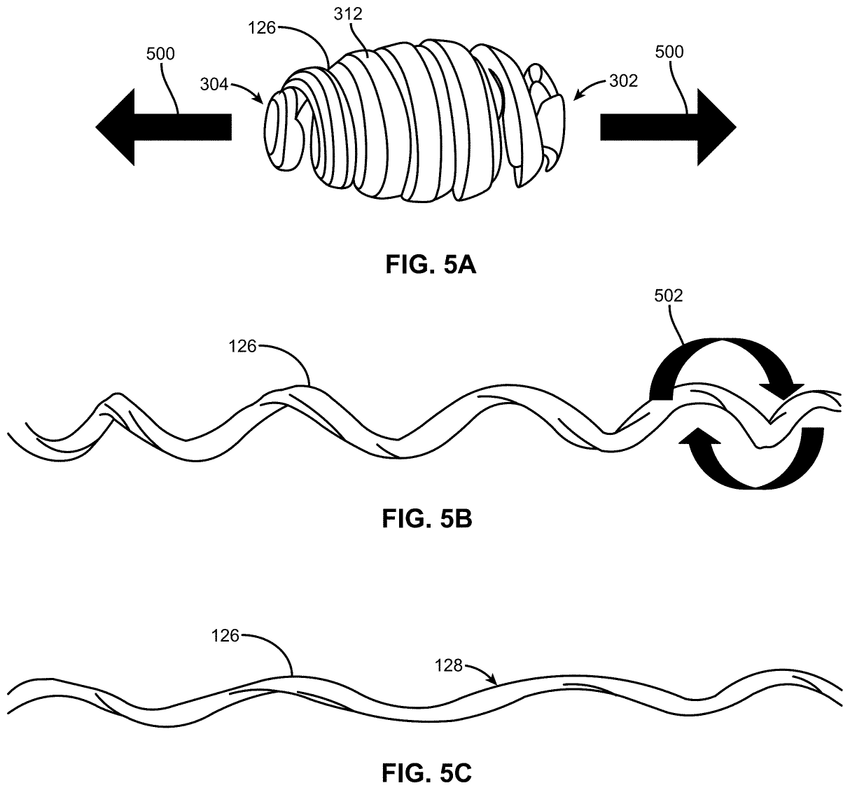

FIG. 5A illustrates the coil member 126 in a relaxed state. The relaxed state can be a state of the coil member 126 when no compressive or tensile forces are applied to the coil member 126. The coil member 126 can be fabricated from or be composed of a biocompatible polymeric material. For example, the coil member 126 can be fabricated from or be composed of silicone, silicone rubber, a thermoplastic elastomer (TPE), or a combination thereof. The coil member 126 can have some elasticity or shape memory characteristics such that the coil member 126 can revert back to its relaxed state or as-molded shape when a force previously applied to the coil member 126 (e.g., a tensile force or a compressive force) ceases to act upon the coil member 126.

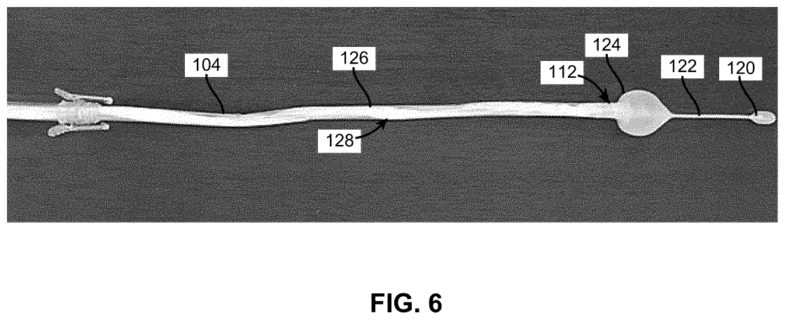

FIGS. 5A-5C illustrate certain steps of a process for preparing the coil member 126 for insertion or positioning within the delivery tube lumen 114 (see FIG. 1, FIG. 7, and FIG. 19). For example, the delivery tube lumen 114 can have a lumen diameter of between approximately 15.0 mm and 17.5 mm. As a more specific example, the delivery tube lumen 114 can have a lumen diameter of about 16.5 mm. Given the small diameter of the delivery tube lumen 114, it is preferable to form the coil member 126 into an elongated narrow configuration 128 (see FIG. 1, FIG. 5C, FIG. 6, and FIG. 7) in order to more easily insert or position the coil member 126 into the delivery tube lumen 114. Moreover, it is also preferable to form the coil member 126 into the elongated narrow configuration 128 to more easily translate (e.g., push, pull, or a combination thereof) the coil member 126 through the delivery tube lumen 114 and into the fillable cavity 400 (see FIG. 8A) of the device covering 124.

FIG. 5A illustrates that the coil member 126 (e.g., the coil distal end 302, the coil proximal end 304, or a combination thereof) can first be pulled in a longitudinal direction 500 to stretch or otherwise form the coil member 126 into an elongated configuration. FIG. 5B illustrates that the coil member 126 can also be rotated a number of full rotations 502 or revolutions to partially or fully unwind the helically wound coils 312. A full rotation 502 can refer to a rotation of about 360°. In some variations, the coil member 126 can be partially unwound by rotating the coil member 126 between approximately two full rotations 502 and three full rotations 502. In other variations, the coil member 126 can be partially unwound by rotating the coil member 126 between approximately three full rotations 502 and five full rotations 502. In additional variations, the coil member 126 can be fully unwound by rotating the coil member 126 between approximately 5.5 full rotations 502 and six full rotations 502. FIG. 5C illustrates the coil member 126 in the elongated narrow configuration 128 after being elongated and partially unwound. Although FIGS. 5A and 5B illustrate the coil member 126 as being elongated first and then rotated, it is contemplated by this disclosure that the coil member 126 can be elongated and rotated simultaneously. Moreover, the coil member 126 can also be elongated after being rotated first.

FIG. 6 is a black-and-white image of a variation of the gastric obstruction device 102 coupled to a delivery tube 104 and an elongated and partially unwound coil member 126 positioned within the delivery tube 104. FIG. 6 illustrates that the delivery tube 104 can be substantially translucent or see-through such that the coil member 126 positioned within the delivery tube lumen 114 is visible to a user or operator. FIG. 6 also illustrates that the coil member 126 in the elongated narrow configuration 128 can occupy or take up a significant amount of space within the delivery tube lumen 114. One benefit of elongating and unwinding the coil member 126 between approximately two full rotations 502 and five full rotations 502 prior to inserting or positioning the coil member 126 within the delivery tube 104 is to improve the delivery or translation of the coil member 126 through and out of the delivery tube lumen 114. For example, the coil member 126 can be more easily translated (e.g., pulled, pushed, or a combination thereof) through the delivery tube lumen 114 when the coil member is partially unwound and elongated. More specifically, unwinding or partially unwinding the coil member 126 can result in less friction exerted on the coil member 126 by the internal wall of the delivery tube 104 as the coil member 126 is translated through and out of the delivery tube 104.

As will be discussed in the following sections, the coil member 126 will need to recover its rotations when the coil member 126 is delivered through and eventually out of the delivery tube lumen 114 into the fillable cavity 400 (see FIG. 8A) of the device covering 124. The coil member 126 will need to recover its rotations in order for the coil member 126 to form into the contracted widened configuration 300 within the fillable cavity 400 (see FIG. 8A) of the device covering 124. For example, the coil member 126 will need to recover its rotations in order for the coil protuberances 402 (see FIG. 4B) of the helically wound coils 312 to nest into neighboring coil furrows 404 (see FIG. 4B).

Although not shown in FIG. 6, a segment of the delivery tube 104 comprising the coil member 126 and the remainder of the gastric obstruction device 102 coupled to the delivery tube distal end 112 can further be inserted into an access sheath. The device covering 124 of the gastric obstruction device 102 can be compressed to fit within a lumen of the access sheath. The access sheath comprising the gastric obstruction device 102 and the segment of the delivery tube 104 comprising the coil member 126 can be introduced per-orally through the oral cavity, pharynx, and esophagus of the patient and a distal segment of the access sheath can be positioned within the stomach of the patient. When at least part of the gastric obstruction device 102 is within the stomach of the patient, the access sheath can be pulled back or unsheathed and the device covering 124, the tether 122, and the distal occluding member 120 can be exposed within the stomach of the patient. At this point, the coil member 126 within the delivery tube lumen 114 can be introduced into the fillable cavity 400 (see FIG. 8A) of the device covering 124.

FIG. 7 illustrates a perspective view of one variation of part of the system 100 for deploying the gastric obstruction device 102. As depicted in FIG. 7, the system 100 can comprise a control tube 700 configured to extend through the delivery tube lumen 114 and engage with a part of the gastric obstruction device 102.

The control tube 700 can be fabricated from or be composed of a biocompatible polymeric material, metallic material or alloy, or a combination thereof. In one variation, the control tube 700 can be fabricated from or be composed of polyether ether ketone (PEEK). In other variations, the control tube 700 can be fabricated from or be composed of fluoropolymers, polycarbonate, stainless steel, or a combination thereof.

In some variations, a segment of the control tube 700 can be an elongate cylinder having a control tube lumen 702. As will be discussed in more detail in the following sections, a plurality of tension lines 1400 (see FIG. 14A, FIG. 14B, and FIG. 14C) can extend or pass through the control tube lumen 702.

The cylindrical segment of the control tube 700 can have a control tube diameter 704. In some variations, the control tube diameter 704 can be between approximately 2.50 mm and 3.50 mm. In other variations, the control tube 700 can have a control tube diameter 704 of between approximately 3.25 mm and 3.50 mm. In additional variations, the control tube 700 can have a control tube diameter 704 of between approximately 3.50 mm and 5.00 mm.

In some variations, the control tube 700 can have a control tube length dimension of between approximately 700 mm and 950 mm. In other variations, the control tube 700 can have a control tube length dimension of between approximately 950 mm and 1200 mm. The differential in size between the control tube length dimension and the control tube diameter 704 can allow the control tube 700 to easily bend or curve.

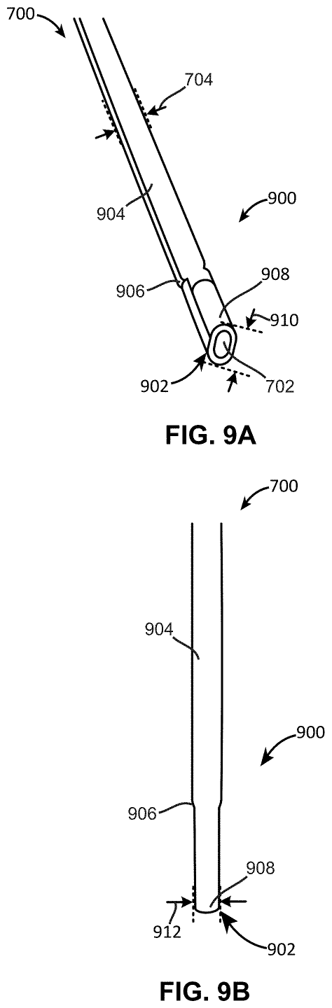

The control tube 700 can comprise a control tube proximal segment 706 and a control tube distal segment 900 (see FIG. 9A and FIG. 9B). As depicted in FIG. 7, the entire coil member 126 in the elongated narrow configuration 128 can be wound around the control tube 700 in a helical manner. For example, the coil member 126 wound around the control tube 700 can be elongated and partially unwound (e.g., unwound approximately two to three full rotations). In some variations, the control tube length dimension can be greater than an end-to-end length of the entire unwound and elongated coil member 126.

A part of the control tube distal segment 900 (see FIG. 9A and FIG. 9B) can extend into the device covering 124 and detachably engage or mate with a part of the gastric obstruction device 102. As will be discussed in the following sections, the control tube distal segment 900 can detachably engage or mate with a lock component 322 of the distal hub 320 coupled to the device covering 124.

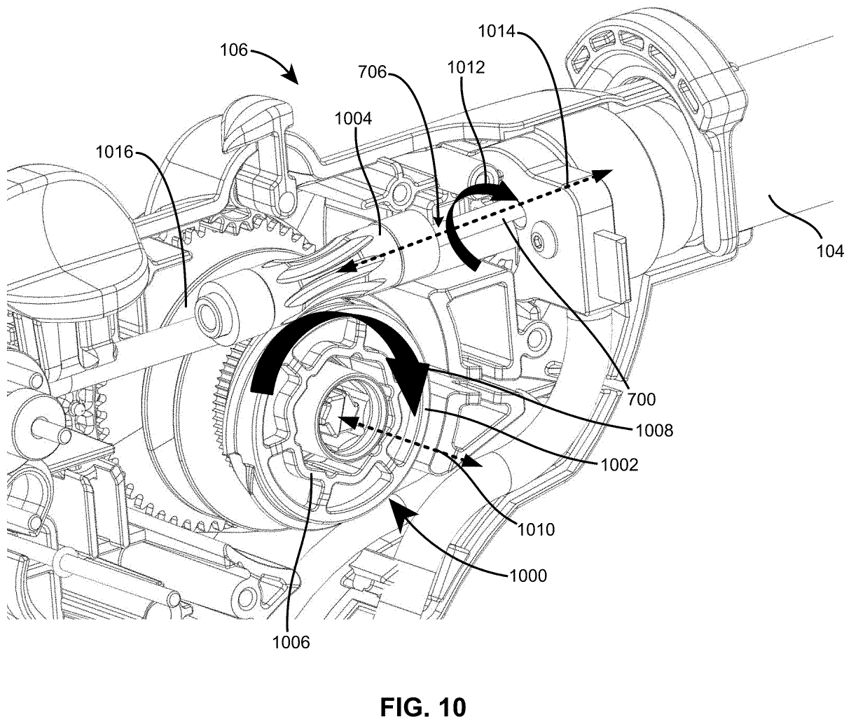

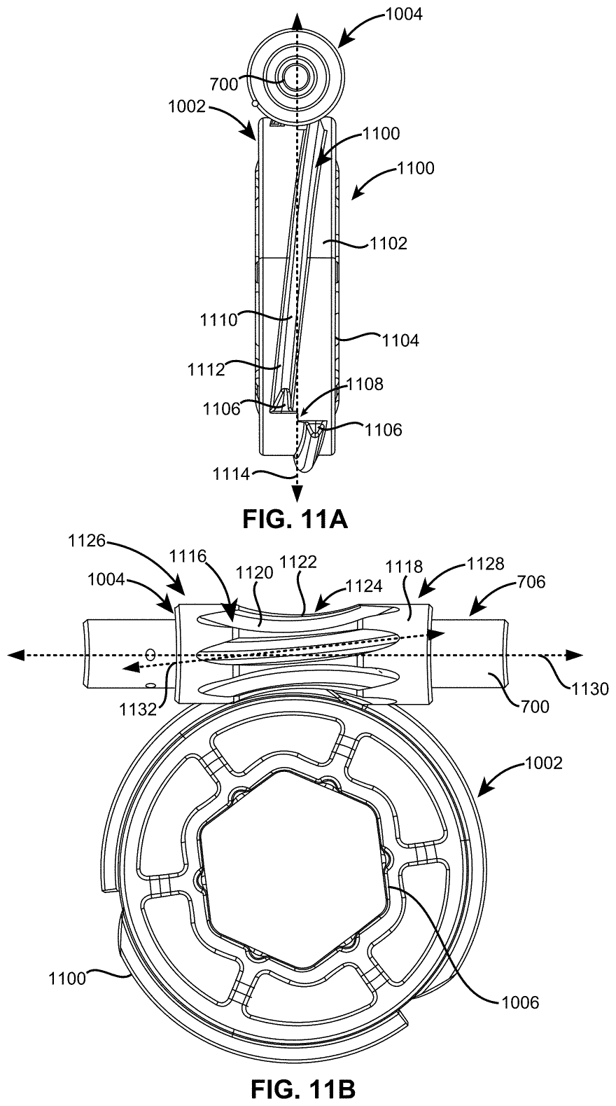

The control tube proximal segment 706 can be positioned within the housing 106 (see FIG. 1). A portion of the control tube proximal segment 706 can be coupled to a part of a gear mechanism 1000 (see FIG. 10). As will be discussed in the following sections, the control component 108 (see FIG. 1 and FIG. 19) can be coupled to a first gear component (e.g., a worm wheel 1002) of the gear mechanism 1000 (see FIG. 10) and a length of the control tube proximal segment 706 can be coupled to a second gear component (e.g., a worm barrel 1004) of the gear mechanism 1000 (see FIG. 10). Rotating the control component 108 can cause the first gear component (e.g., the worm wheel 1002 of FIG. 10, FIG. 11A, or FIG. 11B) to rotate. This rotation of the first gear component (e.g., the worm wheel 1002 of FIG. 10, FIG. 11A, or FIG. 11B) can cause the second gear component (e.g., the worm barrel 1004) to rotate. The rotation of the second gear component (e.g., the worm barrel 1004) can then cause the entire control tube 700 to rotate.

As depicted in FIG. 7, rotation of the control tube 700 can cause the control tube distal segment 900 (see FIG. 9A and FIG. 9B) to rotate when part of the control tube distal segment 900 is mated to the distal hub 320 (see FIG. 8A and FIG. 8B). Rotation of the distal hub 320 can cause the device covering 124 coupled to the distal hub 320 to rotate. As a result, rotation of the control tube 700 can cause the device covering 124 to rotate. Moreover, rotation of the control tube 700 can also cause the tether 122 and the distal occluding member 120 to rotate. Furthermore, rotation of the control tube 700 can

The coil member 126 in the elongated narrow configuration 128 within the delivery tube lumen 114 can be translated (e.g., pulled, pushed, or a combination thereof) through the delivery tube lumen 114 and into the fillable cavity 400 (see FIG. 8A) of the device covering 124. Rotation of the device covering 124 can cause the coil member 126 to rotate and recover the rotations undone during the unwinding process (see FIG. 5B).

The coil member 126 can begin to rotate when the coil distal end 302 enters the fillable cavity 400 of the device covering 124. In some variations, the coil member 126 can begin to rotate when a segment of the coil member 126 in proximity to the coil distal end 302 is pulled over the distal hub 320 and encircle or surround the distal hub 320. In these and other variations, tension lines 1400 (see FIG. 14A, FIG. 14B, and FIG. 14C) passing or extending transversely through the coil member 126 and the distal hub 320 can facilitate rotation of the coil member 126 in response to the rotation of the distal hub 320. In other variations, the coil member 126 can begin to rotate when the inner surface of the device covering 124 exerts frictional forces on the segment of the coil member 126 entering the fillable cavity 400.

FIG. 7 also illustrates that the control tube proximal segment 706 can rotate around a control tube proximal axis of rotation 708. The control tube proximal axis of rotation 708 can be dictated by the rotation of the second gear component (e.g., the worm barrel 1004 of FIG. 10, FIG. 11A, and FIG. 11B). FIG. 7 further illustrates that the device covering 124 can rotate around a device covering axis of rotation 710 (which can be the same as the second axis of rotation 138 shown in FIG. 1). The device covering axis of rotation 710 can be dictated by the rotation of the distal hub 320. The rotation of the distal hub 320 can, in turn, be dictated by the rotation of the control tube distal segment 900 (see FIG. 9A and FIG. 9B). When the delivery tube 104 and the control tube 700 are kept straight (as shown in FIG. 7), the device covering axis of rotation 710 can be the same as the control tube proximal axis of rotation 708. However, when the delivery tube 104 and the control tube 700 are bent into the curved configuration 1900 of FIG. 19 (e.g. for deployment within the esophagus of a patient), the control tube proximal axis of rotation 708 and the device covering axis of rotation 710 can intersect and be non-parallel. In some instances when the delivery tube 104 and the control tube 700 are bent into the curved configuration 1900, the control tube proximal axis of rotation 708 and the device covering axis of rotation 710 can meet at an oblique angle (e.g., obtuse or acute angle) or a substantially perpendicular angle.

FIG. 7 also illustrates that the device covering 124 can rotate when coupled to the delivery tube distal end 112. The device covering 124 can rotate when the delivery tube 104 remains stationary. As will be discussed in the following sections, the device covering 124 can be detachably coupled to the delivery tube distal end 112 by a flange 116 (see FIG. 3 and FIG. 12B) coupled to the delivery tube distal end 112.

FIG. 8A illustrates a perspective cutaway view of the distal hub 320 positioned within the fillable cavity 400 of the device covering 124. As shown in FIG. 8A, the distal hub 320 can be coupled to the device covering 124 by a plurality of internal struts 800 within the fillable cavity 400. The internal struts 800 can extend radially relative to the distal hub 320. In some variations, one end of each of the internal struts 800 can be coupled to the attachment collar 324 at the base of the distal hub 320 and another end of each of the internal struts 800 can be coupled to the interior surface of the device covering 124. For example, one end of the internal struts 800 can be coupled to the attachment collar 324 and another end of the internal struts 800 can be coupled to the interior surface of the device covering 124 along the tapered pyloric contact region 200. In additional variations, the internal struts 800 can be extensions of or integrated with the device covering 124.

The internal struts 800 can allow the distal hub 320 to translate rotational motion to the rest of the device covering 124. In some variations, the internal struts 800 can be fabricated from or be composed of the same material as the device covering 124. For example, the internal struts 800 can be fabricated from or be composed of a biocompatible polymeric material. As a more specific example, the internal struts 800 can be fabricated from silicone, silicone rubber, thermoplastic elastomer (TPE), or a combination thereof.

As illustrated in FIG. 8A, the attachment collar 324 of the distal hub 320 can also be coupled to an interior portion of the cover distal end 306 by fasteners, adhesives, wires, strings, or a combination thereof. The lock component 322 of the distal hub 320 can protrude or extend into the fillable cavity 400 of the device covering 124. The lock component 322 can comprise a mating cavity 802 at the center of the lock component 322. The mating cavity 802 can be a void or opening designed to receive a key portion 908 (see FIG. 9A) of the control tube 700. As depicted in FIG. 8A, the mating cavity 802 can be substantially oval-shaped, oblong-shaped, stadium-shaped, obround-shaped, or a combination thereof. The unique shape of the mating cavity 802 (e.g., oval-shaped, oblong-shaped, stadium-shaped, obround-shaped, or a combination thereof) can allow the key portion 908 (see FIG. 9A) of the control tube 700 to easily mate or enter into the mating cavity 802 and yet allow the control tube 700 to effectively translate torque from one end of the control tube 700 (e.g., the control tube proximal segment 706) to another end of the control tube 700 (e.g., the control tube distal segment 900). The unique shape of the mating cavity 802 (e.g., oval-shaped, oblong-shaped, stadium-shaped, and/or obround-shaped) can also allow the key portion 908 (see FIG. 9A) of the control tube 700 to easily mate or enter into the mating cavity 802 when the control tube 700 is in a curved configuration 1900 (see FIG. 19). Moreover, the unique shape of the mating cavity 802 can also allow the control tube 700 to more effectively translate torque from one end of the control tube 700 to the other when the control tube 700 is in the curved configuration 1900 (see FIG. 19).

The distal hub 320, including the attachment collar 324 and the lock component 322, can be fabricated from or composed of a biocompatible polymeric material, metallic material or alloy, or a combination thereof. In some variations, the distal hub 320, including the attachment collar 324 and the lock component 322, can be fabricated from or composed of polyether ether ketone (PEEK). In other variations, the distal hub 320, including the attachment collar 324 and the lock component 322, can be fabricated from or be composed of fluoropolymers, polycarbonate, stainless steel, or a combination thereof.

FIG. 8B illustrates a top plan view of the distal hub 320 positioned within the device covering 124. In the variation of the gastric obstruction device 102 shown in FIG. 8B, the distal hub 320 can be coupled to the interior surface of the device covering 124 by four internal struts 800 arranged uniformly around the distal hub 320. For example, the four internal struts 800 can be arranged in a cross-shape or X-shape such that each of the four internal struts 800 can be separated from its neighboring internal strut 800 by approximately 90° degrees.

In other variations, the distal hub 320 can be coupled to the device covering 124 by between six and eight internal struts 800. In all such variations, the internal struts 800 can be arranged uniformly around the distal hub 320 and the internal struts 800 can be separated from one another by substantially uniform distances.

FIGS. 8A and 8B illustrate that the lock component 322 can be accessed through the cover opening 310 of the device covering 124. The control tube distal segment 900 (see FIG. 9A) can extend through the cover opening 310 and mate with or key into the lock component 322 within the fillable cavity 400 of the device covering 124. The control tube distal segment 900 (see FIG. 9A) can be mated with or keyed into the lock component 322 when the device covering 124 is secured to the delivery tube distal end 112 by the flange 116 of the delivery tube 104. In addition, a part of the control tube distal segment 900 (see FIG. 9A) can be mated with or keyed into the lock component 322 when the control tube 700 is being rotated by the gear mechanism 1000 (see FIG. 10). The control tube 700 can be detached from the distal hub 320 when the entire coil member 126 is configured into the contracted widened configuration 300 within the fillable cavity 400 of the device covering 124.

Although the coil member 126 is not shown in FIGS. 8A and 8B, it is contemplated by this disclosure that the coil member 126 can form into the contracted widened configuration 300 around the control tube 700. For example, the helically wound coils 312 (see FIG. 3 and FIG. 4B) can wind or loop around the control tube 700 when the coil member 126 is translated into the fillable cavity 400 of the device covering 124. The control tube 700 can finally detach from the distal hub 320 and be retracted out of the fillable cavity 400 of the device covering 124 when the device covering 124 is uncoupled from the delivery tube 104 and the gastric obstruction device 102 is free to move within the stomach of the patient.

FIGS. 9A and 9B illustrate perspective and top plan views, respectively, of a control tube distal segment 900 terminating at a control tube distal end 902. As depicted in FIG. 9A, the control tube distal segment 900 can comprise the end of a cylindrical elongate portion 904 of the control tube 700, a transitional portion 906, and a key portion 908 in between the transitional portion 906 and the control tube distal end 902.

The cylindrical elongate portion 904 can extend from the control tube proximal segment 706 (see FIG. 7) to the transitional portion 906. The cylindrical elongate portion 904 can be approximately 90% to 99% of the length of the control tube 700. As previously discussed, the cylindrical elongate portion 904 can have a control tube diameter 704 of between approximately 2.50 mm and 3.50 mm. In other variations, the cylindrical elongate portion 904 can have a control tube diameter 704 of between approximately 3.25 mm and 3.50 mm. In additional variations, the cylindrical elongate portion 904 can have a control tube diameter 704 of between approximately 3.50 mm and 5.00 mm.

The transitional portion 906 can be a segment of the control tube 700 in between the cylindrical elongate portion 904 and the key portion 908. The transitional portion 906 can be a segment of the control tube 700 where the control tube 700 changes shape and the control tube diameter 704 tapers or decreases in size.

The key portion 908 of the control tube 700 can be a segment of the control tube 700 in between the transitional portion 906 and the control tube distal end 902. The key portion 908 can have a cross-sectional shape or contour compatible with the cross-sectional shape or contour of the mating cavity 802 (see FIG. 8A) of the lock component 322.

For example, the key portion 908 can have a cross-sectional shape or contour similar to the cross-sectional shape or contour of the mating cavity 802 of the lock component 322. The key portion 908 of the control tube 700 can have a slightly smaller cross-sectional shape or contour than the cross-sectional shape or contour of the mating cavity 802 so that the key portion 908 can fit within or enter into the mating cavity 802 (see FIG. 8A and FIG. 8B).

The key portion 908 can have a substantially oval-shaped cross-section, oblong-shaped cross-section, stadium-shaped cross-section, obround-shaped cross-section, or a combination thereof. FIGS. 9A and 9B illustrate that the key portion 908 can have a key height dimension 910 and a key width dimension 912. In some variations, the key height dimension 910 can be between approximately 3.50 mm and 4.50 mm. In other variations, the key height dimension 910 can be between approximately 3.70 mm and 4.10 mm. In some variations, the key width dimension 912 can be between approximately 2.50 mm and 3.50 mm. In other variations, the key width dimension 912 can be between approximately 2.75 mm and 3.10 mm.

Additionally, in some variations, a ratio of the key height dimension 910 to the key width dimension 912 can be between approximately 1.1 and 1.5. In other variations, the ratios of the key height dimension 910 to the key width dimension 912 can be between approximately 1.2 and 1.4. In other variations, the ratios of the key height dimension 910 to the key width dimension 912 can be approximately 1.4.

The device covering 124 (see FIGS. 8A and 8B) can be configured to rotate in response to a rotation of the control tube 700 when the key portion 908 is mated to the lock component 322 of the distal hub 320. As previously discussed with respect to the mating cavity 802 (see FIG. 8A and FIG. 8B), the unique cross-sectional shape of the key portion 908 (e.g., oval-shaped, oblong-shaped, stadium-shaped, obround-shaped, or a combination thereof) can allow the key portion 908 to easily mate with or enter into the mating cavity 802 and yet still allow the control tube 700 to effectively translate torque from one end of the control tube 700 (e.g., the control tube proximal segment 706, see FIG. 7) to another end of the control tube 700 (e.g., the control tube distal segment 900). The unique cross-sectional shape of the key portion 908 (e.g., oval-shaped, oblong-shaped, stadium-shaped, and/or obround-shaped) can also allow the key portion 908 to easily mate with or enter into the mating cavity 802 when the control tube 700 is in a curved configuration 1900 (see FIG. 19). Moreover, the unique cross-sectional shape of the key portion 908 can also allow the control tube 700 to more effectively translate torque from one end of the control tube 700 to the other end when the control tube 700 is in the curved configuration 1900 (see FIG. 19).

Similar to the rest of the control tube, the key portion 908 can be fabricated from or be composed of PEEK. In other variations, the key portion 908 can be fabricated from or be composed of fluoropolymers, polycarbonate, stainless steel, or a combination thereof. In these and other variations, the key portion 908 can be fabricated from or be composed of a different material than the rest of the control tube 700.

FIG. 9A also illustrates that the control tube lumen 702 can extend all the way through the control tube 700 from the control tube proximal segment 706 to the control tube distal segment 900. As will be discussed in the following sections, a number of tension lines 1400 can extend and be pulled through the control tube lumen 702.

The control tube 700 can reversibly or detachably couple with the distal hub 320 when the key portion 908 enters into the mating cavity 802 and becomes secured to the lock component 322 via an interference fit or mechanical fit. The key portion 908 can be mated to the lock component 322 (see FIG. 8A and FIG. 8B) prior to and during the deployment process.

The key portion 908 can be detached or uncoupled from the lock component 322 prior to the device covering 124 being released or removed from the delivery tube 104. The control tube 700 can be decoupled or detached from the distal hub 320 by retracting the key portion 908 from the mating cavity 802.

FIG. 10 illustrates a variation of a gear mechanism 1000 positioned within the housing 106. In some variations, the gear mechanism 1000 can be a worm gear or worm drive mechanism comprising a worm wheel 1002 and a worm barrel 1004. In other variations, the gear mechanism 1000 can be a bevel gear mechanism comprising complementary bevel gear wheels.

The gear mechanism 1000 can comprise a first gear component and a second gear component. In some variations, the first gear component can be or comprise the worm wheel 1002. In these and other variations, the second gear component can be or comprise the worm barrel 1004. As depicted in FIG. 10, the first gear component (e.g., the worm wheel 1002) can be operatively engaged or interlock with the second gear component (e.g., the worm barrel 1004) such that a rotation of the first gear component can cause the second gear component to rotate.

The first gear component (e.g., the worm wheel 1002) and the second gear component (e.g., the worm barrel 1004) can be fabricated from or be composed of a durable polymeric material, metallic material or alloy, or a combination thereof. For example, the first gear component, the second gear component, or a combination thereof can be fabricated from or be composed of nylon, ultra-high-molecular-weight polyethylene (UHMWPE), acetals (e.g., Delrin®) or polyoxymethylenes (POMs), acrylonitrile butadiene styrene (ABS), PTFE, PEEK, phenolic materials, polyesters, polycarbonates, or a combination thereof. In these and other variations, the first gear component, the second gear component, or a combination thereof can be fabricated from or be composed of stainless steel, aluminum, bronze, or a combination thereof.

The first gear component (e.g., the worm wheel 1002) can be coupled to a part of the control component 108. For example, a crankshaft or rod extending from the control component 108 can be operatively engaged, interlocked with, or coupled to a wheel hub 1006 of the first gear component (e.g., the worm wheel 1002). The wheel hub 1006 can comprise an opening or aperture defined in a middle of the first gear component. The wheel hub 1006 can be substantially shaped as a polygon such as a hexagon, an octagon, a decagon, or a combination thereof. The control component 108 can comprise a component configured to key into or mate with the wheel hub 1006. The control component 108 can also be coupled to the first gear component (e.g., the worm wheel 1002) by an interference fit. In addition, fasteners and adhesives can also be used to facilitate coupling of the control component 108 to the first gear component (e.g., the worm wheel 1002).

The control tube 700 can be coupled to the second gear component (e.g., the worm barrel 1004). For example, a portion of the control tube proximal segment 706 can extend through and be affixed to a barrel lumen of the worm barrel 1004. Rotation of the second gear component (e.g., the worm barrel 1004) can result in a rotation of the control tube 700.

The first gear component can be configured to rotate in a first gear rotational direction 1008 around a first gear axis of rotation 1010. For example, the first gear axis of rotation 1010 can be substantially parallel to a directional axis or line extending from one lateral side of the housing 106 to another lateral side of the housing 106. The first gear component can rotate in response to a rotation of the control component 108. The control component 108 can rotate in a rotational direction similar to the first gear rotational direction 1008.

The second gear component can rotate in response to a rotation of the first gear component. For example, the worm barrel 1004 can rotate in response to a rotation of the worm wheel 1002. The second gear component can be configured to rotate in a second gear rotational direction 1012 around a second gear axis of rotation 1014. In some variations, the second gear axis of rotation 1014 can be substantially aligned with a longitudinal axis extending through the control tube 700. The first gear axis of rotation 1010 can be substantially perpendicular to the second gear axis of rotation 1014.

As a more specific example, the worm wheel 1002 can be rotated in a clockwise rotational direction when viewed from the lateral side of the housing 106 coupled to the control component 108 to the other lateral side of the housing 106. In response to the rotation of the worm wheel 1002, the worm barrel 1004 can also be rotated in a clockwise rotational direction when viewed from the control tube proximal segment 706 to the control tube distal segment 900 (see FIG. 9A).

As previously discussed, the control tube distal segment 900 can comprise a key portion 908 (see FIG. 9A) configured to mate or otherwise couple to a lock component 322 of a distal hub 320 within the device covering 124 (see FIG. 8A). Rotation of the control tube 700 can result in a rotation of the gastric obstruction device 102 including the device covering 124, the tether 122, the distal occluding member 120 (see FIG. 1 and FIG. 19), or a combination thereof. Hence, rotation of the control component 108 can result in a rotation of the gastric obstruction device 102 via rotations of the first gear component (e.g., the worm wheel 1002) and the second gear component (e.g., the worm barrel 1004).

A gear ratio can determine the rotation of the second gear component (e.g., the worm barrel 1004) relative to the first gear component (e.g., the worm wheel 1002). In some variations, the first gear component (e.g., the worm wheel 1002) and the second gear component (e.g., the worm barrel 1004) can be configured such that approximately three full rotations (e.g., three 360° rotations or 1080°) of the first gear component (e.g., the worm wheel 1002) can result in approximately one full rotation 502 (e.g., one 360° rotation) of the second gear component (e.g., the worm barrel 1004). In other variations, the first gear component (e.g., the worm wheel 1002) and the second gear component (e.g., the worm barrel 1004) can be configured such that approximately three full rotations (e.g., three 360° rotations or 1080°) of the first gear component (e.g., the worm wheel 1002) can result in approximately two full rotations (e.g., two 360° rotations or 720°) of the second gear component (e.g., the worm barrel 1004). In other variations, the first gear component (e.g., the worm wheel 1002) and the second gear component (e.g., the worm barrel 1004) can be configured such that approximately three full rotations (e.g., three 360° rotations or 1080°) of the first gear component (e.g., the worm wheel 1002) can result in approximately two full rotations (e.g., two 360° rotations or 720°) of the second gear component (e.g., the worm barrel 1004). The gear ratio between the first gear component and the second gear component can also be somewhere in between approximately 3:1 and 3:2.

As previously discussed, the coil member 126 can be partially or fully unwound to facilitate the insertion and delivery of the coil member 126 through the delivery tube 104. The coil member 126 can be unwound by rotating the coil member 126 between approximately two full rotations 502 and six full rotations 502 (see FIGS. 5A, 5B, and 5C). As a more specific example, the coil member 126 can be partially unwound between approximately two full rotations 502 and three full rotations 502. The coil member 126 can recover or regain its rotations when at least part of the coil member 126 enters the fillable cavity 400 of the device covering 124 (see FIG. 8A).

Rotation of the control component 108 can result in a rotation of the gastric obstruction device 102 including a rotation of the device covering 124. In some variations, three rotations of the control component 108 (i.e., three full rotations of the worm wheel 1002) can result in one rotation of the gastric obstruction device 102 or one rotation of the device covering 124 (via a rotation of the worm barrel 1004 and the control tube 700). Moreover, three rotations of the control component 108 (i.e., three full rotations of the worm wheel 1002) can result in one rotation of the coil member 126 as the coil member 126 winds and forms into the contracted widened configuration 300 (see FIG. 4B) within the device covering 124.

Since the coil member 126 can initially be unwound between two full rotations 502 and three full rotations 502 (e.g., 3.5 full rotations 502, see FIGS. 5A, 5B, and 5C), rotating the control component 108 (and thereby the first gear component, e.g., the worm wheel 1002) between approximately six and nine full rotations is needed in order to rotate the device covering 124 and the coil member 126 between approximately two full rotations 502 and three full rotations 502. The number of rotations of the control component 108 and the first gear component can change as the gear ratio between first gear component and the second gear component changes. For example, the control component 108 (and thereby the first gear component, e.g., the worm wheel 1002) can be rotated at least nine full rotations. In this example, the gastric obstruction device 102 including the device covering 124 can be rotated at least three full rotations in order to fully deploy the coil member 126 within the fillable cavity 400 of the device covering 124.

For example, a method of deploying the gastric obstruction device 102 can comprise advancing the gastric obstruction device 102 per-orally into proximity of the stomach of a patient. The gastric obstruction device 102 can be coupled to the control tube distal segment 900 (see FIG. 9A). The control tube proximal segment 706 can be coupled to the worm barrel 1004 of a worm gear. The method can further comprise rotating the control component 108 (see FIG. 1 and FIG. 19) coupled to the worm wheel 1002. The worm wheel 1002 can be rotated in response to the rotation of the control component 108. The worm barrel 1004 can be rotated in response to the rotation of the worm wheel 1002. In addition, the gastric obstruction device 102 can be rotated within the stomach of the patient in response to the rotation of the worm wheel 1002. The method can also comprise rotating the control component 108 at least six to nine full rotations. In other variations, the control component 108 can be rotated such that the gastric obstruction device 102, including the device covering 124, rotates at least three full rotations.

FIG. 10 also illustrates that the gear mechanism 1000 can be coupled to a spool 1016. For example, the spool 1016 can be coupled or extend from the worm wheel 1002. In one variation, the spool 1016 can be integrated with the worm wheel 1002 and be part of one molded component. In other variations, the spool 1016 can be coupled to the worm wheel 1002 by fasteners, linkages, adhesives, shafts, or a combination thereof.

The spool 1016 can rotate in response to a rotation of the first gear component (e.g., the worm wheel 1002) and the control component 108. The spool 1016 can rotate in the same rotational direction as the first gear component. The spool 1016 can be used to reel in certain tension lines 1400 extending through the control tube lumen 702 (see FIG. 7) onto the spool 1016.

FIGS. 11A and 11B illustrate front and side views, respectively, of a first gear component (e.g., the worm wheel 1002) operatively engaged or interlocked with a second gear component (e.g., the worm barrel 1004) of part of the gear mechanism 1000 shown in FIG. 10.

FIG. 11A illustrates that the worm wheel 1002 can comprise a plurality of wheel blades 1100. The wheel blades 1100 can be circumferentially-oriented gear teeth that extend or project radially outward or externally from a circumferential surface 1102 of a wheel disk 1104.

Each of the wheel blades 1100 can terminate at two blade ends 1106. The blade ends 1106 of neighboring or adjacent wheel blades 1100 can be laterally offset 1108 or laterally separated from one another such that the blade ends 1106 do not touch or directly contact one another. In addition, a length dimension of each of the wheel blades 1100 (as measured from one blade end to another blade of the same wheel blade 1100) can be less than a circumference of the wheel disk 1104. This means that each of the wheel blades 1100 only extends partially around the circumference of the wheel disk 1104.

Each of the wheel blades 1100 can also have a blade ridge 1110 (also referred to as a blade top-land) and a blade face 1112. The blade ridge 1110 can run oblique or be aligned at an oblique angle relative to a midline 1114 bisecting the circumferential surface 1102 of the wheel disk 1104. In other words, the blade ridge 1110 can be slanted with respect to the midline 1114. In some variations, the blade ridge 1110 can be curved or twisted. Since the blade ends 1106 of the wheel blades 1100 do not touch and the wheel blades 1100 are slanted, the wheel blades 1100 can appear as disjointed helical protrusions extending radially from the circumferential surface 1102 of the wheel disk 1104.

As depicted in FIG. 11B, a portion of the control tube proximal segment 706 can be coupled to the second gear component. For example, a portion of the control tube proximal segment 706 can extend through and be affixed to a barrel lumen of the worm barrel 1004.

The worm barrel 1004 can comprise a plurality of barrel grooves 1116 that project radially inward from a lateral surface 1118 of the worm barrel 1004. As depicted in FIG. 11B, each of the barrel grooves 1116 can have an arcuate or curved groove surface 1120. The barrel grooves 1116 can also be separated by barrel splines 1122. For example, two neighboring barrel grooves 1116 can flank or be separated by one barrel spline 1122. Similar to the barrel grooves 1116, the barrel splines 1122 can also be curved or have an arcuate contour. In some variations, the barrel splines 1122 do not extend out radially past the lateral surface 1118 of the worm barrel 1004. In these variations, the edges of the barrel splines 1122 are level or flush with the lateral surface 1118 of the worm barrel 1004. In other variations, the barrel splines 1122 can extend out radially past the lateral surface 1118 of the worm barrel 1004.

As shown in FIGS. 10 and 11B, the curvature of the barrel grooves 1116 and the barrel spline 1122 can result in the worm barrel 1004 having a radially convergent midsection 1124. The worm barrel 1004 can be substantially hourglass-shaped due to its radially convergent midsection 1124. The radially convergent midsection 1124 can allow the worm wheel 1002 to more effectively engage with and drive the worm barrel 1004.

The worm barrel 1004 can have a barrel proximal portion 1126 and a barrel distal portion 1128. Each of the barrel grooves 1116 and each of the barrel splines 1122 can be oriented substantially in a longitudinal direction such that each of the barrel grooves 1116 and each of the barrel splines 1122 extends from the barrel proximal portion 1126 to the barrel distal portion 1128.

FIG. 11B also illustrates that the barrel grooves 1116 can be slanted with respect to a barrel longitudinal axis 1130. For example, each of the barrel grooves 1116 can have a groove midline 1132 bisecting the barrel groove 1116. The groove midline 1132 can run oblique to the barrel longitudinal axis 1130 or meet the barrel longitudinal axis 1130 at an oblique angle. The orientation of the barrel grooves 1116 can complement the orientation of the wheel blades 1100 and allow the worm barrel 1004 to more effectively engage with the worm wheel 1002.

The rotation of the worm wheel 1002 can cause at least one of the wheel blades 1100 of the worm wheel 1002 to impart a translational motion to at least one of the groove surfaces 1120 and the barrel splines 1122 to rotate the worm wheel 1002. In some variations, the worm barrel 1004 can be configured to be rotated 360° in response to a rotation of the worm wheel 1002 of 1080°. In other variations, the worm barrel 1004 can be configured to be rotated 720° in response to a rotation of the worm wheel 1002 of 1080°.

FIG. 11B also illustrates that the worm wheel 1002 can have a substantially hexagonal-shaped wheel hub 1006 defined in a middle of the worm wheel 1002. The wheel hub 1006 can be configured to receive a shaft or drive gear coupled to the control component 108 for rotating the worm wheel 1002.

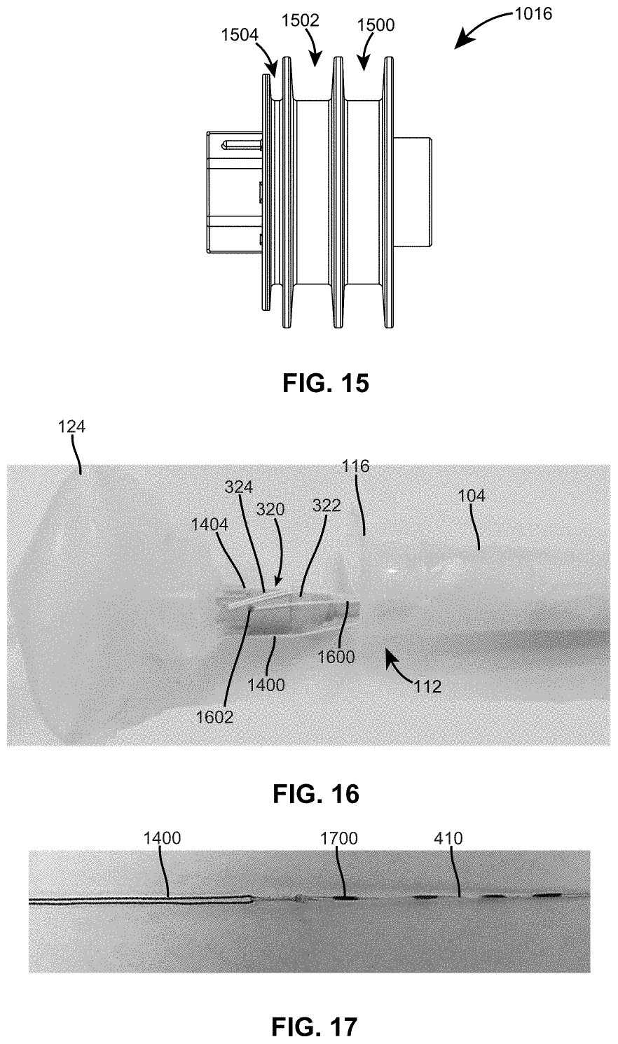

The unique design of the gear mechanism 1000 disclosed herein (including the unique worm wheel 1002 and complementary worm barrel 1004) provides previously undiscovered advantages pertaining to the rotation of an elongate control tube 700 which is, in turn, responsible for the rotation of the unique gastric obstruction device 102 disclosed herein having a fillable device covering 124 (see FIG. 4B and FIG. 8A) and a stretched coil member 126 designed to wind and compress into a contracted widened configuration 300 within the device covering 124. The unique design of the gear mechanism 1000 disclosed herein (including the unique worm wheel 1002 and complementary worm barrel 1004) also provides the added benefit of allowing the control component 108 to be used to control or drive multiple components within the same housing 106. For example, the gear ratio provided by the unique gear mechanism 1000 disclosed herein can allow the control component 108 to control or drive both the worm barrel 1004 and the spool 1016 (see FIG. 10 and FIG. 15) used to reel in the tension lines 1400. Since more rotations of the spool 1016 are needed to reel in the tension lines 1400 than are needed to rotate the device covering 124 (and ultimately, the coil member 126), the gear ratio provided by the unique gear mechanism 1000 disclosed herein allows for one control component 108 to handle this differential in rotations. The unique design of the gear mechanism 1000 disclosed herein (including the unique worm wheel 1002 and complementary worm barrel 1004) provides previously undiscovered advantages pertaining to the safe deployment of a removable gastric obstruction device 102 within the stomach of a patient which is well tolerated by both the stomach and gastrointestinal tract of the patient. The unique gear mechanism 1000 disclosed herein allows the gastric obstruction device 102 to be rotated in a smooth and controlled manner within the stomach of the patient without causing unnecessary trauma to the gastrointestinal tract of the patient.

FIG. 12A illustrates a side cross-sectional view of a variation of a flange 116 uncoupled to a device covering 124 of the gastric obstruction device 102. In some variations, the flange 116 can be coupled to the delivery tube distal end 112. In other variations, the flange 116 can be integrated with the delivery tube 104 and be an extension of the delivery tube 104. The flange 116 can be substantially funnel-shaped, frustoconical-shaped, or a combination thereof. The flange 116 can be soft and compressible such that the flange 116 can radially compress or decrease in size when pushed or pulled through an opening (e.g., the cover opening 310) having a smaller diameter than the maximum diameter of the flange 116.

The flange 116 can be uncoupled to the device covering 124 when the flange 116 has not been inserted into the fillable cavity 400 of the device covering 124 or when the flange 116 has been retracted from the fillable cavity 400 prior to release of the gastric obstruction device 102 from the delivery tube 104. The flange 116 can be inserted into and retracted from the fillable cavity 400 through the cover opening 310 of the device covering 124.

As depicted in FIG. 12A, the cover opening 310 can be circumferentially surrounded by an inwardly curving portion 1200 of the device covering 124. The inwardly curving portion 1200 can be a portion of the device covering 124 at the cover proximal end 308. The inwardly curving portion 1200 can be configured to curve inward or attain a partial funnel-shape by heat treatment or another type of shape-memory treatment. The curvature of the inwardly curving portion 1200 can also be an as-molded shape of this portion of the device covering 124. The inwardly curving portion 1200 can be configured to curve inward or attain a partial funnel-shape when no stress or forces are applied to the cover proximal end 308 of the device covering 124.

FIG. 12A also illustrates that an exterior surface of the flange 116 can be coated or covered by a lubricious coating 1202 to reduce friction between the flange 116 and the interior surface of the device covering 124 when the device covering 124 is rotated while coupled to the flange 116. In some variations, the lubricious coating 1202 can be a polymeric coating or surface treatment. For example, the lubricious coating 1202 can be or comprise a parylene N coating, a parylene C coating, a parylene D coating, or a combination thereof. In other variations, the lubricious coating 1202 can be a dry lubricant such as a dry film lubricant. In other variations, the interior surface of the device covering 124 near the cover opening 310 can also be coated with the lubricious coating 1202.

FIG. 12B illustrates that the flange 116 can facilitate securement of the device covering 124 to the delivery tube 104. The flange 116 can extend through the cover opening 310 of the device covering 124 and secure the device covering 124 by an interference fit. For example, the flange 116 can be momentarily compressed or constricted when it enters through the cover opening 310 and then expand when at least part of the flange 116 is positioned within the fillable cavity 400. Entry of the flange 116 through the cover opening 310 and into the fillable cavity 400 can also draw out or otherwise bias the inwardly curving portion 1200 out of the fillable cavity 400. For example, the inwardly curving portion 1200 can be everted when drawn or biased out of the fillable cavity 400.

The everted or biased inwardly curving portion 1200 can exert a radially inward force on an exterior surface of the flange 116 while the now compressed flange 116 can exert a radially outward force on the interior surface of the device covering 124. Although not shown in FIG. 12B, the control tube 700 (see FIG. 7) can extend through the delivery tube lumen 114 and be mated to the distal hub 320 within the device covering 124. For example, the key portion 908 of the control tube 700 can be mated to the lock component 322 of the distal hub 320 within the device covering 124. The control tube 700 can function to maintain an axial distance between the device covering 124 and the flange 116 such that the device covering 124 does not inadvertently translate proximally with respect to the flange 116.

The device covering 124 can rotate in response to a rotation of the control tube 700. The device covering 124 can rotate when the device covering 124 is coupled to the flange 116. The flange 116 can remain stationary when the device covering 124 is rotated in response to the rotation of the control tube 700. The lubricious coating 1202 on the exterior surface of the flange 116 can reduce the friction between the device covering 124 and the flange 116 when the former is rotated with respect to the latter. The lubricious coating 1202 can also improve the material durability of the flange 116.

The flange 116 can also be configured to substantially seal the cover opening 310 when at least part of the flange 116 extends through the cover opening 310. By sealing the cover opening 310, the flange 116 can create a fluid passageway between the delivery tube lumen 114 and the fillable cavity 400 of the device covering 124. The fluid passageway can be created in order to inflate the fillable cavity 400 of the device covering 124. A fluid can be introduced through the fluid delivery port 130 (see FIG. 1) of the housing 106 and directed through the delivery tube lumen 114 into the fillable cavity 400. In some variations, the fluid can be or comprise an inert gas. In other variations, the fluid can be or comprise a liquid. The fillable cavity 400 of the device covering 124 can be inflated to a relatively low pressure between approximately 0.125 psi and 0.275 psi. As a more specific example, the fillable cavity 400 can be inflated to a pressure of approximately 0.250 psi. Inflating the fillable cavity 400 of the device covering 124 can be done prior to or during the deployment of the coil member 126 into the fillable cavity 400. Inflating the fillable cavity 400 can ensure the device covering 124 is not collapsed or malformed when the coil member 126 is being introduced into the fillable cavity 400. Inflating the fillable cavity 400 can also increase the torsional rigidity or stiffness of the device covering 124 such that the device covering 124 can rotate without becoming twisted.

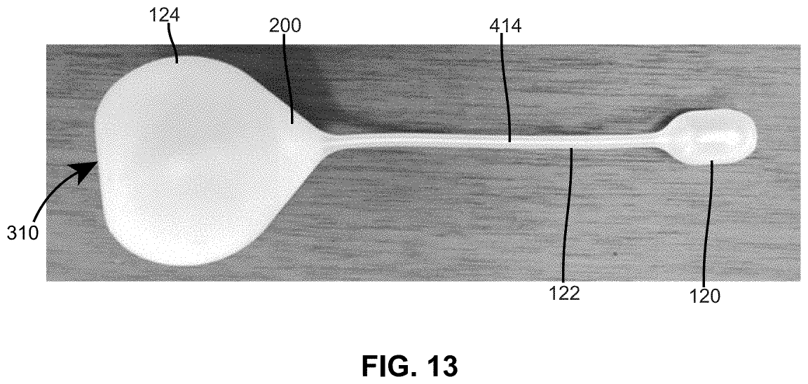

FIG. 13 is a black-and-white image of a variation of part of the gastric obstruction device 102 comprising a device covering 124, a tether 122, and a distal occluding member 120. The inwardly curving portion 1200 is not shown in FIG. 13 since the delivery tube 104 and flange 116 are uncoupled from the device covering 124 and the inwardly curving portion 1200 is curved into the fillable cavity 400. The inwardly curving portion 1200 can exhibit a tendency to recover its inwardly curving shape as soon as the flange 116 is removed from the fillable cavity 400 of the device covering 124.

FIG. 14A is a black-and-white image of tension lines 1400 extended through the helically wound coils 312 of the coil member 126 and into the distal hub 320 coupled to the device covering 124. The device covering 124 shown in FIGS. 14A to 14C has been everted and drawn back in order to more clearly show the distal hub 320 normally positioned within the fillable cavity 400 (see FIG. 8A) of the device covering 124. Moreover, the delivery tube 104 has been omitted from FIGS. 14A to 14C to more clearly show the tension lines 1400 extending through the helically wound coils 312 of the coil member 126. When positioned within the delivery tube lumen 114 (see FIG. 1 and FIG. 19), the coil member 126 would appear more like the coil member 126 shown in FIG. 6 (i.e., in the elongated narrow configuration 128) rather than the coil member 126 shown in FIGS. 14A to 14C.

The tension lines 1400 can be wires, strings, or other types of connecting members used to pull the coil member 126 through the delivery tube 104 and into the fillable cavity 400 of the device covering 124. In some variations, the tension lines 1400 can be fabricated from or be composed of biocompatible high-strength fibers including any number of synthetic polymeric fibers. For example, the tension lines 1400 can be fabricated from or be composed of medical-grade nylon, polyester fiber, polyvinylidene fluoride (PVDF) fiber, UHMWPE fiber, polypropylene fiber, or a combination thereof. In other variations, the tension lines 1400 can be fabricated from or be composed of stainless steel wires.

The tension lines 1400 can extend transversely through the helically wound coils 312 of the coil member 126. The tension lines 1400 can extend or pass through bores 1402 defined through each of the helically wound coils 312. The bores 1402 can be openings or channels defined through each of the helically wound coils 312.

The tension lines 1400 can enter into one or more openings 1404 arranged circumferentially around the attachment collar 324 of the distal hub 320 and loop around within an interior of the distal hub 320 and head in a reverse direction within the distal hub 320. The tension lines 1400 can then enter into the control tube lumen 702 (see FIG. 14B) of the control tube 700 mated with or otherwise coupled to the distal hub 320.

The tension lines 1400 can be pulled in a direction indicated by directional arrows 1406. For example, the tension lines 1400 can first be pulled distally in a direction of the device covering 124 and then be pulled proximally toward the housing 106 (see FIG. 1 and FIG. 19) once the tension lines 1400 have reversed course within the distal hub 320.

The tension lines 1400 can be reeled into the housing 106 by the spool 1016 coupled to the gear mechanism 1000 (see FIG. 10). For example, rotating the control component 108 (see FIG. 1 and FIG. 19) can cause the spool 1016 to rotate and reel in the tension lines 1400. In other variations, the tension lines 1400 can also be pulled or translated through other means besides the spool 1016.

FIG. 14A also illustrates that the tension lines 1400 can be coupled to lock lines 410 at a terminal end of the tension lines 1400. The lock lines 410 can be a separate set of wires, strings, or connecting members configured to remain within the coil member 126 when the coil member 126 is formed into the contracted widened configuration 300 (see FIG. 3, FIG. 4B, and FIG. 18C) to lock or otherwise maintain the coil member 126 in the contracted widened configuration 300. The lock lines 410 will be discussed in more detail in the following sections.

Although FIG. 14A illustrates the tension lines 1400 as loosened or in a relaxed configuration, tension can be applied to the tension lines 1400 during the deployment of the gastric obstruction device 102 such that the tension lines 1400 are taut or tightened when the tension lines 1400 are pulled distally through the delivery tube lumen 114 (see FIG. 7) and proximally through the control tube lumen 702. In addition, although one pair of tension lines 1400 are shown in FIGS. 14A to 14C, it is contemplated by this disclosure that between four and eight pairs of tension lines 1400 can be arranged circumferentially and uniformly around the helically wound coils 312 of the coil member 126 such that different circumferential segments of the coil member 126 are advanced uniformly through the delivery tube lumen.

FIG. 14B is a black-and-white image of the tension lines 1400 extending into the distal hub 320 and exiting out of the lock component 322 of the distal hub 320 into the control tube lumen 702. The control tube 700 is separated from the distal hub 320 in FIG. 14B so as to more clearly show the entry of the tension lines 1400 into the control tube lumen 702. While the coil member 126 is being translated through the delivery tube lumen 114 during deployment of the gastric obstruction device 102, the control tube distal segment 900 (including the key portion 908 of FIG. 9A) would be mated with the lock component 322 of the distal hub 320 and the tension lines 1400 would enter into the control tube lumen 702 without having to exit the distal hub 320.

FIGS. 14A and 14B also illustrate that a segment of the coil member 126 at the coil distal end 302 is configured to fit around the distal hub 320 such that the segment of the coil member 126 at the coil distal end 302 encircles and rings around the distal hub 320 when the coil member 126 forms into the contracted widened configuration 300 within the fillable cavity 400 of the device covering 124. For example, the segment of the coil member 126 at the coil distal end 302 can be substantially shaped as a loop or circle having a break or fracture along the loop or circle so as to more easily allow the loop or circle to fit around the distal hub 320.

FIG. 14C is a black-and-white image of the tension lines 1400 exiting the control tube lumen 702 at a proximal end of the control tube 700. The segment of the tension lines 1400 exiting the control tube lumen 702 can be reeled onto the spool 1016 within the housing 106. Once the coil member 126 has been locked into the contracted widened configuration 300 within the fillable cavity 400 of the device covering 124, the tension lines 1400 can be cut or severed such that the tension lines 1400 can be separated from the lock lines 410 and removed from the control tube lumen 702.

FIG. 15 illustrates a variation of a spool 1016 configured to rotate in response to a rotation of the control component 108. The spool 1016 can be coupled to part of the gear mechanism 1000 of FIG. 10. For example, the spool 1016 can be coupled to or extend from the worm wheel 1002. The spool 1016 can rotate in response to a rotation of the first gear component (e.g., the worm wheel 1002) and the control component 108. The spool 1016 can rotate in the same rotational direction as the first gear component.

In some variations, the spool 1016 can be fabricated from or be composed of nylon, acrylonitrile butadiene styrene (ABS), ultra-high-molecular-weight polyethylene (UHMWPE), acetals (e.g., Delrin®) or polyoxymethylenes (POMs), PTFE, PEEK, phenolic materials, polyesters, polycarbonates, or a combination thereof. In other variations, the spool 1016 can also be fabricated from or be composed of stainless steel, aluminum, bronze, or a combination thereof.

The spool 1016 can be used to reel in a plurality of tension lines 1400 extending through the delivery tube lumen 114 and the control tube lumen 702 (see FIG. 7) onto the spool 1016. The spool 1016 can also be used to reel in one or more anchor lines 1600 (see FIG. 16) configured to secure or moor the gastric obstruction device 102 to the system 100 during the deployment of the gastric obstruction device 102.

As depicted in FIG. 15, the spool 1016 can comprise a first tension line partition 1500, a second tension line partition 1502, and an anchor line partition 1504. The first tension line partition 1500 and the second tension line partition 1502 can be used to reel different sets of tension lines 1400 on to different parts of the spool 1016. The first tension line partition 1500 and the second tension line partition 1502 can ensure that different sets of tension lines 1400 extending through different passageways defined by bores 1402 (see FIG. 14A) along the coil member 126 do not become entangled or intertwined when reeled in by the spool 1016. Separating certain of the tension lines 1400 from other tension lines 1400 on the spool 1016 can also allow a clinician or user of the system 100 to cut certain tension lines 1400 while allowing other tension lines 1400 to remain in place. Moreover, separating the anchor line 1600 from the tension lines 1400 can reduce the likelihood that the anchor line 1600 becomes entangled or intertwined with the tension lines 1400.

Although one spool 1016 is shown FIG. 15, it should be understood that multiple spools 1016 (e.g., between two and six spools 1016) can be positioned within the housing 106. The multiple spools 1016 can be configured to reel in different sets of tension lines 1400 extending through the delivery tube lumen 114 and the control tube lumen 702 (see FIG. 7).

FIG. 16 is a black-and-white image of a plurality of tension lines 1400 and an anchor line 1600 extending into the distal hub 320 of the gastric obstruction device 102. Similar to FIGS. 14A to 14C, the device covering 124 shown in FIG. 16 has been everted and drawn back in order to more clearly show the distal hub 320 normally positioned within the fillable cavity 400 (see FIG. 8A) of the device covering 124.

The anchor line 1600 can be made of the same material as the tension lines 1400. For example, the anchor line 1600 can be fabricated from or be composed of medical grade nylon, polyester fiber, polyvinylidene fluoride (PVDF) fiber, UHMWPE fiber, polypropylene fiber, or a combination thereof. In other variations, the anchor line 1600 can be fabricated from or be composed of stainless steel wires.

The anchor line 1600 can be configured to prevent the distal hub 320 of the gastric obstruction device 102 from inadvertently separating from the control tube 700 during the deployment of the gastric obstruction device 102 (including the formation of the coil member 126 within the fillable cavity 400 of the device covering 124). The anchor line 1600 can enter an anchor line aperture 1602 defined along the attachment collar 324 of the distal hub 320. The anchor line 1600 can then loop around within the interior of the distal hub 320 and head in a reverse direction within the distal hub 320. Similar to the tension lines 1400, the anchor line 1600 can then enter into the control tube lumen 702 (see FIG. 14B) of the control tube 700 mated with or otherwise coupled to the distal hub 320.

The anchor line 1600 can be pulled in a direction similar to the one shown by the directional arrows 1406 in FIGS. 14A to 14C. For example, the anchor line 1600 can first be pulled distally in a direction of the device covering 124 and then be pulled proximally toward the housing 106 (see FIG. 1 and FIG. 19) once the anchor line 1600 has reversed course within the distal hub 320.

The anchor line 1600 can be reeled into the housing 106 by the spool 1016 coupled to the gear mechanism 1000 (see FIG. 10). The anchor line 1600 can be reeled onto the anchor line partition 1504 of the spool 1016. For example, rotating the control component 108 (see FIG. 1 and FIG. 19) can cause the spool 1016 to rotate and reel in the anchor line 1600. In other variations, the anchor line 1600 can also be pulled or translated through other means besides the spool 1016. Once the coil member 126 has been locked into the contracted widened configuration 300 within the fillable cavity 400 of the device covering 124 (see FIGS. 4B. and 18C), the anchor line 1600 can be cut or otherwise severed and the cut anchor line 1600 can be pulled proximally out of the control tube lumen 702.

FIG. 17 is a black-and-white image of a pair of tension lines 1400 coupled to lock lines 410. The lock lines 410 can be made of the same material as the tension lines 1400. The lock lines 410 can be fabricated from or be composed of a biocompatible polymeric fiber. For example, the lock lines 410 can be fabricated from or be composed of medical-grade nylon, polyester fiber, polyvinylidene fluoride (PVDF) fiber, UHMWPE fiber, polypropylene fiber, or a combination thereof.

As depicted in FIG. 17, the lock lines 410 can have a plurality of color-differentiated segments 1700 along a length of the lock lines 410. The color-differentiated segments 1700 can be segments of the lock lines 410 colored differently than the remainder of the lock lines 410 or the tension lines 1400. For example, the color-differentiated segments 1700 can be red-colored or alternate between red and white. The color-differentiated segments 1700 can be used to inform a clinician or user that the string, wire, or connecting member currently under visual observation (e.g., seen under an endoscope) is in fact a segment of a lock line 410. The color-differentiated segments 1700 can also be used to inform the clinician or user of the progress of the deployment (e.g., whether the coil member 126 has been locked into the contracted widened configuration 300).

FIGS. 18A-18B illustrate that the system 100 can comprise a plunger 1800 configured to be translated through the delivery tube lumen 114 as part of the deployment of the gastric obstruction device 102. For example, the plunger 1800 can be translated distally from the delivery tube proximal end 110 (see FIG. 1 and FIG. 19) to the delivery tube distal end 112 to push or otherwise bias the proximal assembly 314 (including the release mechanism 316 and the proximal plug 318, see FIG. 3) and the coil proximal end 304 toward the device covering 124. The plunger 1800 can be coupled to a plunger rod 1802 configured to be translated proximally and distally through the delivery tube lumen 114. The plunger rod 1802 can be translated via a gear mechanism (such as the gear mechanism 1000 of FIG. 10), a ratchet mechanism, a slide bar, or a combination thereof coupled to a proximal end of the plunger rod 1802 contained within the housing 106. In some variations, the plunger 1800 can be a substantially cylindrical mass for pushing or otherwise advancing the proximal assembly 314 and the coil proximal end 304 distally through the delivery tube lumen 114. In other variations, the plunger 1800 can comprise a substantially funnel-shaped flange at the distal end of the plunger 1800.

FIG. 18A also illustrates that the coil member 126 can comprise a proximal loop 1804 at the coil proximal end 304. The coil proximal loop 1804 can be an enclosed hole or opening defined at the coil proximal end 304. A portion of the proximal assembly 314 can extend through the proximal loop 1804 to lock the coil member 126 in the contracted widened configuration 300. For example, a distal portion of the proximal assembly 314 can extend through the proximal loop 1804 and into a lumen defined by the wound coil member 126 when the proximal assembly 314 and the coil proximal end 304 enter the fillable cavity 400 of the device covering 124 through the cover opening 310 (see FIG. 18B).

The plunger 1800 can push or otherwise translate the proximal assembly 314 and the coil proximal end 304 distally through the delivery tube lumen 114 while the remainder of the coil member 126 is also being pulled through the delivery tube lumen 114 by the tension lines 1400. Moreover, the plunger 1800 can push or otherwise translate the proximal assembly 314 and the coil proximal end 304 distally through the delivery tube lumen 114 while the device covering 124 and the distal hub 320 are being rotated by the rotation of the control tube 700.

FIG. 18C illustrates a separation of the delivery tube 104 from the deployed gastric obstruction device 102 once the coil member 126 is locked into the contracted widened configuration 300 within the device covering 124. As shown in FIG. 18C, the control tube 700 can be uncoupled from the distal hub 320 and retracted proximally out of the delivery tube lumen 114. In addition, the flange 116 can be retracted from the fillable cavity 400 of the device covering 124 once the coil member 126 is locked into the contracted widened configuration 300. The flange 116 can be retracted by pushing or otherwise applying a force (e.g., by an endoscope) to the device covering 124 to dislodge the device covering 124 from the flange 116. At this point, the anchor line 1600 (see FIG. 16) and any tension lines 1400 can be cut or severed such that only the lock lines 410 remain within the wound coil member 126.

In addition, the inwardly curving portion 1200 of the device covering 124 surrounds the cover opening 310 can recover its inwardly curving shape as soon as the flange 116 is removed from the fillable cavity 400. The proximal assembly 314 can then act as a cap or stopper to partially occlude the cover opening 310

At this point, the gastric obstruction device 102 can freely move within the stomach of the patient. Once the patient has ingested food or liquids, the stomach of the patient can begin to contract and relax, repeatedly, such that the distal occluding member 120 is propelled or otherwise moved by peristaltic waves through the stomach towards the pylorus. The tapered pyloric contact region 200 of the gastric obstruction device 102 can then intermittently cover or obstruct the pylorus when at least part of the distal occluding member 120 is within the duodenum of the patient. This intermittent obstruction of the pylorus can cause food and/or liquids to pass from the stomach into the duodenum at a slower rate, thus inducing the patient to feel full sooner and reduce the patient's craving for more food.

FIG. 19 illustrates a perspective view of another variation of the system 100 for deploying the gastric obstruction device 102. FIG. 19 does not depict the coil member 126 in the elongated narrow configuration 128 (see FIG. 1 and FIG. 7) within the delivery tube lumen 114 in order to more clearly show the control tube 700 within the delivery tube lumen 114.

As depicted in FIG. 19, the delivery tube 104 and the control tube 700 can be bent or otherwise formed into a curved configuration 1900. The delivery tube 104 and the control tube 700 can be bent or otherwise formed into the curved configuration 1900 when extended per-orally into the esophagus or stomach of the patient through the oral cavity and the pharynx. The delivery tube 104 and the control tube 700 can be extended into the esophagus and stomach of the patient in order to advance the gastric obstruction device 102 into the stomach of the patient. Bending or curving the delivery tube 104 and the control tube 700 is needed in order to accommodate the natural curvature or tortuosity of the aforementioned organs serving as the delivery path within the patient.

As shown in FIG. 19, the control component 108 can be rotated in the first rotational direction 132 when the delivery tube 104 and the control tube 700 are in the curved configuration 1900. The control component 108 can be rotated in the first rotational direction 132 around the first axis of rotation 134. The control component 108 can be rotated when at least part of the gastric obstruction device 102 is within the stomach of the patient. Rotation of the control component 108 can cause the control tube 700 within the delivery tube lumen 114 to rotate via the gear mechanism 1000 (see FIG. 10) within the housing 106. The control tube 700 can be rotated when in the curved configuration 1900.

Rotation of the control tube 700 can cause the distal hub 320 (see FIG. 8A) mated, attached, or otherwise coupled to the key portion 908 of the control tube distal segment 900 (see FIG. 9A) to rotate. The distal hub 320 can be coupled to the device covering 124 of the gastric obstruction device 102 via the plurality of internal struts 800 (see FIG. 8A and FIG. 8B). The device covering 124 of the gastric obstruction device 102 can then rotate in response to the rotation of the distal hub 320.

As a result, the gastric obstruction device 102 coupled to the delivery tube distal end 112 can rotate in the second rotational direction 136 in response to the rotation of the control component 108 in the first rotational direction 132. The gastric obstruction device 102 can rotate in the second rotational direction 136 around the second axis of rotation 138.

The first axis of rotation 134 can be non-parallel to the second axis of rotation 138 when the delivery tube 104 and the control tube 700 are in the curved configuration 1900. For example, when the delivery tube 104 and the control tube 700 are in the curved configuration 1900, the first axis of rotation 134 can be oblique (e.g., obtuse or acute) to the second axis of rotation 138. More specifically, the first axis of rotation 134 can meet the second axis of rotation 138 at an oblique angle (e.g., an obtuse or acute angle).

A method of operating, preparing, and/or inspecting the gastric obstruction device 102 can comprise providing a distal segment of a delivery tube 104 coupled to the gastric obstruction device 102. A proximal segment of the delivery tube 104 can be coupled to a housing 106. The method can further comprise actuating a control component 108 coupled to the housing 106 in a first rotational direction around a first axis of rotation. The gastric obstruction device 102 coupled to the delivery tube 104 can rotate in a second rotational direction around a second axis of rotation in response to the rotation of the control component 108. The first axis of rotation can be non-parallel to the second axis of rotation.

Another method of operating, preparing, and/or inspecting the gastric obstruction device 102 can comprise providing the gastric obstruction device 102. The gastric obstruction device 102 can be coupled to a distal end of a control tube 700. A proximal end of the control tube 700 can be coupled to a worm barrel of a worm gear. The method can further comprise actuating a control component 108 coupled to a worm wheel of the worm gear. The worm wheel can be rotated in response to the rotation of the control component. The worm barrel can be rotated in response to the rotation of the worm wheel. The gastric obstruction device 102 can be rotated in response to the rotation of the worm wheel.

A number of embodiments have been described. Nevertheless, it will be understood by one of ordinary skill in the art that various changes and modifications can be made to this disclosure without departing from the spirit and scope of the embodiments. Elements of systems, devices, apparatus, and methods shown with any embodiment are exemplary for the specific embodiment and can be used in combination or otherwise on other embodiments within this disclosure. For example, the steps of any methods depicted in the figures or described in this disclosure do not require the particular order or sequential order shown or described to achieve the desired results. In addition, other steps operations may be provided, or steps or operations may be eliminated or omitted from the described methods or processes to achieve the desired results. Moreover, any components or parts of any apparatus or systems described in this disclosure or depicted in the figures may be removed, eliminated, or omitted to achieve the desired results. In addition, certain components or parts of the systems, devices, or apparatus shown or described herein have been omitted for the sake of succinctness and clarity.

Accordingly, other embodiments are within the scope of the following claims and the specification and/or drawings may be regarded in an illustrative rather than a restrictive sense.

Each of the individual variations or embodiments described and illustrated herein has discrete components and features which may be readily separated from or combined with the features of any of the other variations or embodiments. Modifications may be made to adapt a particular situation, material, composition of matter, process, process act(s) or step(s) to the objective(s), spirit or scope of the present invention.

Methods recited herein may be carried out in any order of the recited events that is logically possible, as well as the recited order of events. Moreover, additional steps or operations may be provided or steps or operations may be eliminated to achieve the desired result.

Furthermore, where a range of values is provided, every intervening value between the upper and lower limit of that range and any other stated or intervening value in that stated range is encompassed within the invention. Also, any optional feature of the inventive variations described may be set forth and claimed independently, or in combination with any one or more of the features described herein. For example, a description of a range from 1 to 5 should be considered to have disclosed subranges such as from 1 to 3, from 1 to 4, from 2 to 4, from 2 to 5, from 3 to 5, etc. as well as individual numbers within that range, for example 1.5, 2.5, etc. and any whole or partial increments therebetween.

All existing subject matter mentioned herein (e.g., publications, patents, patent applications) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise, all technical and scientific terms used herein have the same meaning as commonly understood by one of ordinary skill in the art to which this invention belongs.

Reference to the phrase “at least one of”, when such phrase modifies a plurality of items or components (or an enumerated list of items or components) means any combination of one or more of those items or components. For example, the phrase “at least one of A, B, and C” means: (i) A; (ii) B; (iii) C; (iv) A, B, and C; (v) A and B; (vi) B and C; or (vii) A and C.

In understanding the scope of the present disclosure, the term “comprising” and its derivatives, as used herein, are intended to be open-ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Also, the terms “part,” “section,” “portion,” “member” “element,” or “component” when used in the singular can have the dual meaning of a single part or a plurality of parts. As used herein, the following directional terms “forward, rearward, above, downward, vertical, horizontal, below, transverse, laterally, and vertically” as well as any other similar directional terms refer to those positions of a device or piece of equipment or those directions of the device or piece of equipment being translated or moved.

Finally, terms of degree such as “substantially”, “about” and “approximately” as used herein mean the specified value or the specified value and a reasonable amount of deviation from the specified value (e.g., a deviation of up to ±0.1%, ±1%, ±5%, or ±10%, as such variations are appropriate) such that the end result is not significantly or materially changed. For example, “about/approximately 1.0 m” can be interpreted to mean “1.0 m” or between “0.9 m and 1.1 m.” When terms of degree such as “about” or “approximately” are used to refer to numbers or values that are part of a range, the term can be used to modify both the minimum and maximum numbers or values.

This disclosure is not intended to be limited to the scope of the particular forms set forth, but is intended to cover alternatives, modifications, and equivalents of the variations or embodiments described herein. Further, the scope of the disclosure fully encompasses other variations or embodiments that may become obvious to those skilled in the art in view of this disclosure.

1.-30. (canceled)

31. A system for deploying a gastric obstruction device, comprising:
a housing comprising a worm gear, wherein the worm gear comprises a worm wheel and a worm barrel configured to rotate in response to a rotation of the worm wheel;
a control component coupled to the worm wheel; and
a control tube comprising a control tube proximal segment and a control tube distal segment,
wherein the control tube proximal segment is coupled to the worm barrel,
wherein the gastric obstruction device is coupled to the control tube distal segment, and
wherein the gastric obstruction device is configured to rotate in response to a rotation of the control component.

32. The system of claim 31, further comprising a delivery tube comprising a delivery tube proximal end, a delivery tube distal end, and delivery tube lumen in between the delivery tube proximal end and the delivery tube distal end, wherein the delivery tube is coupled to the housing at the delivery tube proximal end and the delivery tube distal end is configured to couple to the gastric obstruction device.

33. The system of claim 32, wherein the control tube is configured to extend through the delivery tube lumen, wherein each of the delivery tube and the control tube is bendable into a curved configuration, and wherein the control tube is configured to rotate when in the curved configuration within the delivery tube.

34. The system of claim 31, wherein the control tube is fabricated from a biocompatible polymeric material.

35. The system of claim 31, wherein the worm wheel comprises a plurality of wheel blades that extend radially outward from a circumferential surface of a wheel disk, wherein the worm barrel comprises a plurality of barrel grooves that project radially inward from a lateral surface of the worm barrel to define a plurality of groove surfaces, and wherein the rotation of the worm wheel causes at least one of the wheel blades to impart a translational motion to at least one of the groove surfaces to rotate the worm barrel.

36. The system of claim 31, wherein the worm wheel comprises a plurality of wheel blades that project radially from a circumferential surface of a wheel disk, wherein each of the wheel blades has a blade ridge and the blade ridge is aligned at an oblique angle relative to a midline bisecting the circumferential surface of the wheel disk, and wherein a length dimension of each of the wheel blades is less than a circumference of the wheel disk.

37. The system of claim 31, wherein the worm barrel comprises a barrel proximal portion and a barrel distal portion, where the worm barrel further comprises a plurality of barrel grooves that project radially inward from a lateral surface of the worm barrel, wherein each of the barrel grooves is oriented substantially in a longitudinal direction such that each of the barrel grooves extend from the barrel proximal portion to the barrel distal portion.

38. The system of claim 31, wherein the worm barrel comprises a radially convergent midsection.

39. The system of claim 31, wherein the worm barrel is configured to be rotated 360 degrees in response to a rotation of the worm wheel of 1080 degrees.

40. The system of claim 31, wherein the worm barrel comprises a barrel lumen and a segment of the control tube extends into the worm barrel.

41. The system of claim 31, wherein the housing further comprises a spool and the worm wheel is coupled to the spool, wherein the control tube further comprises a control tube lumen and a plurality of tension lines extend through the control tube lumen, and wherein the rotation of the control component is configured to reel in the tension lines extending through the control tube lumen.

42. A method of deploying a gastric obstruction device, comprising:
advancing the gastric obstruction device per-orally into proximity of a stomach of a patient,
wherein the gastric obstruction device is coupled to a distal end of a control tube,
wherein a proximal end of the control tube is coupled to a worm barrel of a worm gear;
actuating a control component coupled to a worm wheel of the worm gear,
wherein the worm wheel is rotated in response to the rotation of the control component,
wherein the worm barrel is rotated in response to the rotation of the worm wheel, and
wherein the gastric obstruction device is rotated within the stomach of the patient in response to the rotation of the worm wheel.

43. The method of claim 42, wherein the worm wheel rotates around a first gear axis of rotation, wherein the worm barrel rotates around a second gear axis of rotation, and wherein the first gear axis of rotation is substantially perpendicular to the second gear axis of rotation.

44. The method of claim 42, wherein actuating the control component comprises rotating the control component at least nine full rotations.

45. The method of claim 42, wherein actuating the control component comprises rotating the control component between six and nine full rotations.

46. The method of claim 42, further comprising bending the control tube into a curved configuration in order to advance the gastric obstruction device per-orally into the stomach of the patient, wherein the control tube is rotated in response to the rotation of the worm barrel, and wherein the control tube is in the curved configuration when rotated.

47. The method of claim 42, further comprising coupling the control tube to the gastric obstruction device by mating a key portion of a control tube distal segment with a lock component within the gastric obstruction device.

48. The method of claim 47, wherein the gastric obstruction device further comprises a device covering coupled to the lock component and wherein the device covering rotates in response to the rotation of the control component.

49. The method of claim 48, wherein the gastric obstruction device further comprises a coil member detachably coupled to the device covering and wherein the coil member rotates in response to the rotation of the control component.

50. The method of claim 42, further comprising retracting the control tube from the gastric obstruction device prior to removing the control tube from an esophagus of the patient.

51. (canceled)

52. (canceled)
